(19) United States
(12) Patent Application Publication
(10) Pub. No.: US 2016/0122692 A1
(43) Pub. Date: May 5, 2016

(54) METHOD OF LAUNDERING A FABRIC

(71) Applicant: The Procter & Gamble Company, Cincinnati, OH (US)

(72) Inventors: Neil Joseph LANT, Newcastle upon Tyne (GB); Lindsay Suzanne BEWICK, Tyne & Wear (GB); Steven George PATTERSON, Tyne & Wear (GB); Linsey Sarah FULLER, Newcastle upon Tyne (GB)

(21) Appl. No.: 14/828,589

(22) Filed: Aug. 18, 2015

(30) Foreign Application Priority Data
Aug. 19, 2014 (EP) 14002883.8
Aug. 13, 2015 (WO) US2015/045052

(51) Int. Cl. C11D 3/386; C12N 9/20

(52) U.S. Cl. CPC C11D 3/38627; C11D 3/38681; C12N 9/20; C12Y 301/01003

(57) ABSTRACT

A method of laundering a fabric, comprising the steps of: (i) contacting a fabric with an aqueous wash liquor comprising a detergent composition, the detergent composition comprising a surfactant and wherein the wash liquor comprises from 0.05 to 4 g/l of a surfactant; and (ii) in a first rinse step, contacting the fabric from step (i) with a rinse liquor; (iii) in a second rinse step, contacting the fabric from step (ii) with a second rinse liquor comprising a lipid esterase; (iv) drying the fabric; (v) contacting the fabric from step (iv) with a soil; and (vi) contacting the fabric from step (v) with an aqueous wash liquor of a detergent composition wherein the detergent composition comprises a surfactant, the wash liquor comprising from 0.05 to 4 g/l of a surfactant.

### FIELD OF THE INVENTION

The present invention relates to methods of laundering fabrics.

### BACKGROUND OF THE INVENTION

Fabric care compositions are often added by consumers to the rinse step of a fabric washing operation. Fabric care compositions impart a number of sensorial benefits that consumers enjoy, including softness and freshness. Most frequently, softness is provided by esterified cationic surfactants or silicones. It is also known to add cellulase enzymes into such compositions for example as described in WO95/05442.

Laundry detergent compositions are used to provide fabric cleaning benefits in the wash step of a laundry operation. There is a further need for a means to improve cleaning particularly at low wash temperatures and also at short wash cycles.

The present invention alleviates this problem.

### SUMMARY OF THE INVENTION

The present invention is to a method of laundering a fabric, comprising the steps of;
i) contacting a fabric with an aqueous wash liquor comprising a detergent composition, the detergent composition comprising a surfactant and wherein the wash liquor comprises from 0.05 to 4 g/l of a surfactant; and
ii) in a first rinse step, contacting the fabric from step (i) with a first rinse liquor;
iii) in a second rinse step, contacting the fabric from step (ii) with a second rinse liquor comprising a lipid esterase;
iv) drying the fabric;
v) contacting the fabric from step (iv) with a soil;
vi) contacting the fabric from step (v) with an aqueous wash liquor of a detergent composition wherein the detergent composition comprises a surfactant, the wash liquor comprising from 0.05 to 4 g/l of a surfactant.

Typically the first and second rinse liquors are aqueous. Preferably the rinse liquor, in particular the second rinse liquor additionally comprises a soil release polymer.

### DETAILED DESCRIPTION OF THE INVENTION

The method of the present invention comprises the steps of:
i) contacting a fabric with an aqueous wash liquor comprising a detergent composition, the detergent composition comprising a surfactant and wherein the wash liquor comprises from 0.05 to 4 g/l of a surfactant; and
ii) in a first rinse step, contacting the fabric from step (i) with an aqueous rinse liquor;
iii) in a second rinse step, contacting the fabric from step (ii) with a second aqueous rinse liquor comprising a lipid esterase;
iv) drying the fabric;
v) contacting the fabric from step (iv) with a soil;
vi) contacting the fabric from step (v) with an aqueous wash liquor of a detergent composition wherein the detergent composition comprises a surfactant, the wash liquor comprising from 0.05 to 4 g/l of a surfactant. In step (i) and/or step (vi) it may be preferred for the ratio of surfactant to fabric on a weight to weight basis to be from 1:150 to 1:500 in step (i) and/or step (iii).

In the method of the present invention, in step (i) the fabric may be washed in a conventional wash step in which an aqueous wash liquor is formed by the addition of a detergent composition to water. The surfactant concentration in the aqueous wash liquor is from 0.05 to 4 g/l. In step (ii) the fabric undergoes a rinse step of a wash operation, to substantially remove the wash liquor from the fabric. In step (iii) a further rinse step is provided in which the fabric is contacted with a lipid esterase. The fabric is then dried in step (iv) and undergoes soiling in step (v), i.e. the fabric may then be worn by a consumer or used in another way for its intended use. Following use of the fabric, the fabric may then be contacted with a further aqueous wash liquor in step (vi). Without wishing to be bound by theory, it is believed that the lipid esterase contacted to the fabric in step (iii) acts ‘out of the wash’ to hydrolyse the soils/stains which contact the fabric during the soiling step (v). This leads to good soil removal in a subsequent wash step (vi) and effective stripping of soil from the fabric in step (vi). This can also be used to enable a reduction in the amount of surfactant used to provide effective cleaning.

### Step (i)

The method of the present invention comprises a step (i) of contacting the fabric with an aqueous wash liquor. The aqueous wash liquor is formed by the addition of a laundry detergent composition to water. The detergent composition added to water to form the aqueous wash liquor may be in any suitable form including granular, liquid or unitized dose. When in unitized dose form, it is preferred that the composition is enclosed within a water-soluble film, for example a polyvinyl alcohol-based film. The fabric may be contacted with the composition in a hand washing step or even a wash liquor in a machine wash cycle.

The laundry detergent composition typically comprises from 1 to 70 wt %, or from 2 to 50 wt % or from 5 to 40 wt %, based on the total weight of the laundry detergent composition, of a surfactant. The concentration of the surfactant in the wash liquor is from 0.05 to 5 g/l, or from 0.1 to 4 g/l.

The detersive surfactant may be an anionic, cationic, non-ionic, zwitterionic, amphoteric surfactant or a combination thereof. The surfactant composition may comprise one surfactant or typically mixtures of more than one surfactant.

Preferred anionic detersive surfactants are alkyl benzene sulfonates, alkoxylated anionic surfactant, or a combination thereof. Suitable anionic detersive surfactants include sulphate and sulphonate detersive surfactants.

Particularly preferred alkyl benzene sulphonates are linear alkylbenzene sulphonates, particularly those having a carbon chain length of C8-15, or C₁₀₋₁₃ alkyl benzene sulphonate.

Suitable alkyl benzene sulphonate (LAS) is obtainable, or even obtained, by sulphonating commercially available linear alkyl benzene (LAB); suitable LAB includes low 2-phenyl LAB, such as those supplied by Sasol under the tradename Isochem® or those supplied by Petresa under the tradename Petrelab®, other suitable LAB include high 2-phenyl LAB, such as those supplied by Sasol under the tradename Hyblene®. Another suitable anionic detersive surfactant is alkyl benzene sulphonate that is obtained by DETAL catalyzed process, preferably having 8 to 15 carbon atoms. Other synthesis routes, such as HF, may also be suitable.

Suitable sulphate detersive surfactants include alkyl sulphate, such as C₈₋₁₈ alkyl sulphate, or predominantly C₁₂ alkyl sulphate. The alkyl sulphate may be derived from natural sources, such as coco and/or tallow. Alternatively, the alkyl sulphate may be derived from synthetic sources such as C₁₂₋₁₅ alkyl sulphate.

It may be preferred for the surfactant composition to comprise in addition an alkyl alkoxylated sulphate, such as alkyl ethoxylated sulphate, or a C₈₋₁₈ alkyl alkoxylated sulphate, or a C₈₋₁₈ alkyl ethoxylated sulphate. Preferably the alkyl chain length may be from 12 to 16 carbon atoms. The alkyl alkoxylated sulphate may have an average degree of alkoxylation of from 0.5 to 20, or from 0.5 to 10, or from 0.5 to 7, or from 0.5 to 5 or from 0.5 to 3. Examples include predominantly C12 sodium lauryl ether sulphate ethoxylated with an average of 3 moles of ethylene oxide per mole.

The alkyl sulphate, alkyl alkoxylated sulphate and alkyl benzene sulphonates may be linear or branched, substituted or un-substituted.

The anionic detersive surfactant may be a mid-chain branched anionic detersive surfactant, such as a mid-chain branched alkyl sulphate and/or a mid-chain branched alkyl benzene sulphonate. The mid-chain branches are typically C₁₋₄ alkyl groups, such as methyl and/or ethyl groups.

Another suitable anionic detersive surfactant is alkyl ethoxy carboxylate.

The anionic surfactants are typically present in their salt form, typically being complexed with a suitable cation. Suitable counter-ions include Na⁺ and K⁺, substituted ammonium such as C₁-C₆ alkanolammnonium such as mono-ethanolamine (MEA) tri-ethanolamine (TEA), di-ethanolamine (DEA), and any mixture thereof.

Preferably the surfactant composition comprises a non-ionic detersive surfactant in addition to the anionic surfactant. Preferred nonionic surfactants are primary and secondary alcohol alkoxylates, especially ethoxylates. Suitable non-ionic detersive surfactants include alkyl alkoxylated alcohols, such as C₈₋₁₈ alkyl alkoxylated alcohol, or a C₈₋₁₈ alkyl ethoxylated alcohol. The alkyl alkoxylated alcohol may have an average degree of alkoxylation of from 0.5 to 50, or from 1 to 30, or from 1 to 20, or from 1 to 10. The alkyl alkoxylated alcohol may be a C₈₋₁₈ alkyl ethoxylated alcohol, typically having an average degree of ethoxylation of from 1 to 10, or from 1 to 7, or from 1 to 5, or from 3 to 7. The alkyl alkoxylated alcohol can be linear or branched, and substituted or un-substituted.

Suitable examples of nonionic surfactants include those selected from the group consisting of: C₈-C₁₈ alkyl ethoxylates, such as, NEODOL® non-ionic surfactants from Shell; C₆-C₁₂ alkyl phenol alkoxylates wherein optionally the alkoxylate units are ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols; C₁₄-C₂₂ mid-chain branched alkyl alkoxylates, typically having an average degree of alkoxylation of from 1 to 30; alkylpolysaccharides, such as alkylpolyglycosides; polyhydroxy fatty acid amides; ether capped poly(oxyalkylated) alcohol surfactants; and mixtures thereof. Suitable non-ionic detersive surfactants are also alkyl polyglucoside and/or an alkyl alkoxylated alcohol.

Suitable nonionic detersive surfactants include secondary alcohol-based detersive surfactants having the formula:

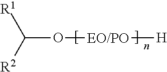

wherein R¹=linear or branched, substituted or unsubstituted, saturated or unsaturated C₂₋₈ alkyl; wherein R²=linear or branched, substituted or unsubstituted, saturated or unsaturated C₂₋₈ alkyl, wherein the total number of carbon atoms present in R¹+R² moieties is in the range of from 7 to 13; wherein EO/PO are alkoxy moieties selected from ethoxy, propoxy, or mixtures thereof, optionally the EO/PO alkoxyl moieties are in random or block configuration; wherein n is the average degree of alkoxylation and is in the range of from 4 to 10.

Other suitable non-ionic detersive surfactants include EO/PO block co-polymer surfactants, such as the Plurafac® series of surfactants available from BASF, and sugar-derived surfactants such as alkyl N-methyl glucose amide.

The ratio of anionic surfactant to nonionic surfactant may be from 2:1 to 1:2, or even from 1:1 to 1:3 or from greater than 1:1 to 1:2.

The composition may also comprise an amine oxide preferably in amounts up to 10 wt % of the surfactant composition. Suitable amine oxides are described in WO2014/114570, a particularly preferred amine oxide comprising lauryl dimethylamine oxide. The composition may also comprise a zwitterionic surfactant. A preferred zwitterionic surfactant is a betaine surfactant, for example a carbobetaine, such as Empigen® from Huntsman. Where amine oxide and/or betaine surfactant is present, the weight ratio of anionic and/or nonionic surfactant to amine oxide and/or betaine is typically from 10:1 to 20:1.

The laundry detergent composition may comprise a lipid esterase. The lipid esterase can be any lipid esterase. The lipid esterase may be a lipase, or a cutinase, or a combination thereof.

The lipid esterase may be selected from the following:
(1) Triacylglycerol lipases (E.C. 3.1.1.3)
(2) Carboxylic ester hydrolase (E.C. 3.1.1.1)
(3) Cutinase (E.C. 3.1.1.74)
(4) Sterol esterase (E.C. 3.1.1.13)
(5) Wax-ester hydrolase (E.C. 3.1.1.50)

Suitable triacylglycerol lipases can be selected from variants of the Humicola lanuginosa (Thermomyces lanuginosus) lipase. Other suitable triacylglycerol lipases can be selected from variants of Pseudomonas lipases, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), Bacillus lipases, e.g., from B. subtilis (Dartois et al. (1993), Biochemica et Biophysica Acta, 1131, 253-360), B. stearothermophilus (JP 64/744992) or B. pumilus (WO 91/16422).

Suitable carboxylic ester hydrolases can be selected from wild-types or variants of carboxylic ester hydrolases endogenous to B. gladioli, P. fluorescens, P. putida, B. acidocaldarius, B. subtilis, B. stearothermophilus, Streptomyces chrysomallus, S. diastatochromogenes and Saccaromyces cerevisiae.

Suitable cutinases can be selected from wild-types or variants of cutinases endogenous to strains of Aspergillus, in particular Aspergillus oryzae, a strain of Alternaria, in particular Alternaria brassiciola, a strain of Fusarium, in particular Fusarium solani, Fusarium solani pisi, Fusarium oxysporum, Fusarium oxysporum cepa, Fusarium roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular Rhizoctonia solani, a strain of Streptomyces, in particular Streptomyces scabies, a strain of Coprinopsis, in particular Coprinopsis cinerea, a strain of Thermobifida, in particular Thermobifida fusca, a strain of Magnaporthe, in particular Magnaporthe grisea, or a strain of Ulocladium, in particular Ulocladium consortiale.

In a preferred embodiment, the cutinase is selected from variants of the Pseudomonas mendocina cutinase described in WO 2003/076580 (Genencor), such as the variant with three substitutions at I178M, F180V, and S205G.

In another preferred embodiment, the cutinase is a wild-type or variant of the six cutinases endogenous to Coprinopsis cinerea described in H. Kontkanen et al, App. Environ. Microbiology, 2009, p 2148-2157

In another preferred embodiment, the cutinase is a wild-type or variant of the two cutinases endogenous to Trichoderma reesei described in WO2009007510 (VTT).

In a most preferred embodiment the cutinase is derived from a strain of Humicola insolens, in particular the strain Humicola insolens DSM 1800. Humicola insolens cutinase is described in WO 96/13580 which is hereby incorporated by reference. The cutinase may be a variant, such as one of the variants disclosed in WO 00/34450 and WO 01/92502. Preferred cutinase variants include variants listed in Example 2 of WO 01/92502. Preferred commercial cutinases include Novozym 51032 (available from Novozymes, Bagsvaerd, Denmark).

Suitable sterol esterases may be derived from a strain of Ophiostoma, for example Ophiostoma piceae, a strain of Pseudomonas, for example Pseudomonas aeruginosa, or a strain of Melanocarpus, for example Melanocarpus albomyces.

In a most preferred embodiment the sterol esterase is the Melanocarpus albomyces sterol esterase described in H. Kontkanen et al, Enzyme Microb Technol., 39, (2006), 265-273.

Suitable wax-ester hydrolases may be derived from Simmondsia chinensis. The lipid esterase may be selected from an enzyme in E.C. class 3.1 or 3.2 or a combination thereof. The lipid esterase may be selected from an enzyme in E.C. class 3.1.1.1 or 3.1.1.3 or a combination thereof.

The detergent composition from step (i) optionally contains one or more additional detergent ingredients as described below.

The fabric may be any suitable fabric. The fabric may comprise natural or synthetic materials or a combination thereof. The fabric may comprise cotton, polycotton, polyester, or a combination thereof. The fabric may comprise cotton.

### Step (ii)

In step (ii) the washed fabric from step (i) undergoes a rinse step, typically using an aqueous rinse liquor. This can be a hand rinsing step in which fabric from step (i) is placed into rinse liquor, that is substantially free from the detergent composition present in step (i), or a rinse step in a washing machine. Typically, subsequently rinse liquor is removed from the fabric by conventional means. Step (ii) may include two or more separate rinse stages in which fresh rinse liquor is provided in respective stages. Although rinsing does not remove all of the laundry detergent composition from a wash step, step (ii) aims to at least partially, preferably substantially, remove the laundry detergent composition from step (i) from the fabric.

Step (iii)

The method of the present invention comprises a second rinse step (iii) in which the fabric is contacted with a second rinse liquor comprising a lipid esterase.

The lipid esterase may be any suitable lipid esterase. The lipid esterase may comprise at least a first and a second lipid esterase. The lipid esterase may comprise more than two lipid esterases. The lipid esterase may be a lipase, or a cutinase, or a combination thereof.

The lipid esterase may be selected from the following:

(1) Triacylglycerol lipases (E.C. 3.1.1.3)

(2) Carboxylic ester hydrolase (E.C. 3.1.1.1)

(3) Cutinase (E.C. 3.1.1.74)

(4) Sterol esterase (E.C. 3.1.1.13)

(5) Wax-ester hydrolase (E.C. 3.1.1.50)

By ‘E.C. class’ we herein mean the Enzyme Commission class. The Enzyme Commission class is an international recognized enzyme classification scheme based on chemical reactions that the enzymes catalyse.

Suitable triacylglycerol lipases can be selected from variants of the Humicola lanuginosa (Thermomyces lanuginosus) lipase. Other suitable triacylglycerol lipases can be selected from variants of Pseudomonas lipases, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), Bacillus lipases, e.g., from B. subtilis (Dartois et al. (1993), Biochemica et Biophysica Acta, 1131, 253-360), B. stearothermophilus (JP 64/744992) or B. pumilus (WO 91/16422).

Suitable carboxylic ester hydrolases can be selected from wild-types or variants of carboxylic ester hydrolases endogenous to B. gladioli, P. fluorescens, P. putida, B. acidocaldarius, B. subtilis, B. stearothermophilus, Streptomyces chrysomallus, S. diastatochromogenes and Saccaromyces cerevisiae.

Suitable cutinases can be selected from wild-types or variants of cutinases endogenous to strains of Aspergillus, in particular Aspergillus oryzae, a strain of Alternaria, in particular Alternaria brassiciola, a strain of Fusarium, in particular Fusarium solani, Fusarium solani pisi, Fusarium oxysporum, Fusarium oxysporum cepa, Fusarium roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular

Rhizoctonia solani, a strain of Streptomyces, in particular Streptomyces scabies, a strain of Coprinopsis, in particular Coprinopsis cinerea, a strain of Thermobifida, in particular Thermobifida fusca, a strain of Magnaporthe, in particular Magnaporthe grisea, or a strain of Ulocladium, in particular Ulocladium consortiale.

In a preferred embodiment, the cutinase is selected from variants of the Pseudomonas mendocina cutinase described in WO 2003/076580 (Genencor), such as the variant with three substitutions at I178M, F180V, and S205G.

In another preferred embodiment, the cutinase is a wild-type or variant of the six cutinases endogenous to Coprinopsis cinerea described in H. Kontkanen et al, App. Environ. Microbiology, 2009, p 2148-2157

In another preferred embodiment, the cutinase is a wild-type or variant of the two cutinases endogenous to Trichoderma reesei described in WO2009007510 (VTT).

In a most preferred embodiment the cutinase is derived from a strain of Humicola insolens, in particular the strain Humicola insolens DSM 1800. Humicola insolens cutinase is described in WO 96/13580 which is hereby incorporated by reference. The cutinase may be a variant, such as one of the variants disclosed in WO 00/34450 and WO 01/92502. Preferred cutinase variants include variants listed in Example 2 of WO 01/92502.

Suitable sterol esterases may be derived from a strain of Ophiostoma, for example Ophiostoma piceae, a strain of Pseudomonas, for example Pseudomonas aeruginosa, or a strain of Melanocarpus, for example Melanocarpus albomyces.

In a most preferred embodiment the sterol esterase is the Melanocarpus albomyces sterol esterase described in H. Kontkanen et al, Enzyme Microb Technol., 39, (2006), 265-273.

Suitable wax-ester hydrolases may be derived from Simmondsia chinensis.

The lipid esterase may be selected from an enzyme in E.C. class 3.1 or 3.2 or a combination thereof. The lipid esterase may comprise an enzyme selected from E.C. class 3.1.1.1 or 3.1.1.3 or 3.1.1.74 or a combination thereof. The preferred lipid esterase may comprise an enzyme selected from E.C. class 3.1.1.3. The lipid esterase may comprise a variant having at least 90% sequence identity to wild-type lipase from Thermomyces lanuginosus and having sequence substitutions T231R and N233R, or a variant corresponding to claim 5, part (u) of EP1290150B1, or a combination thereof. The lipid esterase may comprise a variant having at least 90% sequence identity to wild-type lipase from Thermomyces lanuginosus and having sequence substitutions T231R and N233R.

The fabric may be contacted with a lipid esterase at a concentration of between 30 and 55,000 ng enzyme/g fabric. The fabric may be contacted with a lipid esterase at a concentration of between 30 and 2000 ng enzyme/g fabric. The fabric may be contacted with a lipid esterase at a concentration of between 50 and 1700 ng enzyme/g fabric, or even 80 and 1600 ng enzyme/g fabric. The fabric may be contacted with a lipid esterase at a concentration of between 100 and 3000 ng enzyme/g fabric, or even 125 and 2500 ng enzyme/g fabric. The fabric may be contacted with the lipid esterase at a concentration of between 100 and 35,000 ng enzyme/g fabric, or even between 500 and 30,000 ng enzyme/g fabric. Without wishing to be bound by theory, it is believed that these concentrations are optimal for soil removal from the fabrics.

In step (ii) the fabric from step (ii) is contacted with the lipid esterase in a second rinse step. This can be a hand rinsing step in which fabric from step (ii) is placed into rinse water, that is substantially free from the detergent composition present in step (i), or a rinse step in a washing machine. Step (iii) contacts the fabric with lipid esterase thereby depositing the lipid esterase onto the fabric.

The rinse liquor of the second rinse step may comprise optional additional rinse-added ingredients, such as fabric softening ingredients, such as quaternary ammonium surfactants or silicones and optionally other ingredients useful in the rinse stage, such as perfumes, dyes, polymers, surfactants.

Preferably the aqueous rinse liquor from the second rinse step (iii) comprises a soil release polymer. The soil release polymer may be present in the aqueous rinse liquor in amounts from 0.00001 to 3 g/l in the rinse liquor, or from 0.0001 to 2 g/l or up to 1 g/l or 0.5 g/l. The rinse liquor may be provided by adding a fabric softening composition to water to for the aqueous rinse liquor. The soil release polymer may be added via the fabric softening composition. In such fabric softening composition, the soil release polymer may be present in amounts from about 0.01% to about 10.0%, typically from about 0.1% to about 5%, in some aspects from about 0.2% to about 3.0%, by weight of the composition, of a soil release polymer (also known as a polymeric soil release agents or “SRA”).

Suitable soil release polymers typically have hydrophilic segments to hydrophilize the surface of hydrophobic fibers, such as polyester and nylon, and hydrophobic segments to deposit on hydrophobic fibers and remain adhered thereto through completion of washing and rinsing cycles, thereby serving as an anchor for the hydrophilic segments. This may enable stains occurring subsequent to treatment with a soil release agent to be more easily cleaned in later washing procedures.

Soil release agents may include a variety of charged, e.g., anionic or cationic (see, e.g., U.S. Pat. No. 4,956,447), as well as non-charged monomer units. The structure of the soil release agent may be linear, branched, or star-shaped. The soil release polymer may include a capping moiety, which is especially effective in controlling the molecular weight of the polymer or altering the physical or surface-active properties of the polymer. The structure and charge distribution of the soil release polymer may be tailored for application to different fibers or textile types and for formulation in different detergent or detergent additive products. Suitable polyester soil release polymers have a structure as defined by one of the following structures (III), (IV) or (V):

—[(OCHR¹—CHR²)ₐ—O—OC—Ar—CO—]_{d} (III)

—[(OCHR³CHR⁴)_{b}—O—OC-sAr—CO]ₑ (IV)

—[(OCHR⁵—CHR⁶), OR⁷]_{f} (V)

wherein:
a, b and c are from 1 to 200;
d, e and f are from 1 to 50;
Ar is a 1,4-substituted phenylene;
sAr is 1,3-substituted phenylene substituted in position 5 with SO₃Me;
Me is H, Na, Li, K, Mg+2, Ca+2, Al+3, ammonium, mono-, di-, tri-, or tetra-alkylammonium wherein the alkyl groups are C1-C18 alkyl or C2-C10 hydroxyalkyl, or any mixture thereof;
R¹, R², R³, R⁴, R⁵ and R⁶ are independently selected from H or C, —C18 n- or iso-alkyl; and R⁷ is a linear or branched C1-C18 alkyl, or a linear or branched C2-C30 alkenyl, or a cycloalkyl group with 5 to 9 carbon atoms, or a C6-C30 aryl group, or a C6-C30 arylalkyl group.

Suitable polyester soil release polymers are terephthalate polymers having the structure (III) or (IV) above. Other suitable soil release polymers may include, for example sulphonated and unsulphonated PET/POET polymers, both end-capped and non-end-capped. Examples of suitable polyester soil release polymers are the REPEL-O-TEX® line of polymers supplied by Rhodia, including REPEL-O-TEX® SRP6 and REPEL-O-TEX® SF-2. Other suitable soil release polymers include TexCare® polymers, including TexCare® SRA-100, TexCare® SRA-300, TexCare® SRN-100, TexCare® SRN-170, TexCare® SRN-240, TexCare® SRN-300, and TexCare® SRN-325, all supplied by Clariant. Especially useful soil release polymers are the sulphonated non-end-capped polyesters described in WO 95/32997A (Rhodia Chimie) Other suitable soil release polymers are Marloquest® polymers, such as Marloquest® SL supplied by Sasol. Examples of SRAs are described in U.S. Pat. Nos. 4,968,451; 4,711,730; 4,721,580; 4,702,857; 4,877,896; 3,959,230; 3,893,929; 4,000,093; 5,415,807; 4,201,824; 4,240,918; 4,525,524; 4,201,824; 4,579,681; and 4,787,989; European Patent Application 0 219 048; 279,134 A; 457,205 A; and DE 2,335,044; and WO201419792; WO2012104156/57/58, WO201419658; WO20141965; WO201429479.

Optionally the second rinse liquor additionally comprises a fabric softener active selected from the group comprising, diester quaternary ammonium compounds, dialkyl quaternary ammonium compounds, imidazolinium quaternary compounds, cationic starch, sucrose ester-based fabric care materials, and mixtures thereof. In one aspect, said ester quat fabric softener active, monoester, diester, and triester quat fabric softener active and ion pair fabric softener actives are selected from the group consisting of:
a) materials having Formula (1) below

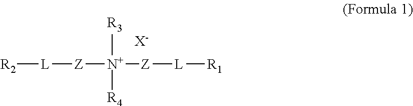

wherein:
(i) R₁ and R₂ are each independently a C₅-C₂₃ hydrocarbon;
(ii) R₃ and R₄ are each independently selected from the group consisting of C₁-C₄ hydrocarbon, C₁-C₄ hydroxy substituted hydrocarbon, benzyl, —(C₂H₄O)_{y}H where y is an integer from 1 to 10;
(iii) L is selected from the group consisting of —C(O)O—, —OC(O)—;
(iv) Z is selected from the group consisting of —(CH₂)ₙ, —CH₂C(CH₃)H— where each n is independently an integer from 1 to 4
(v) X⁻ is a softener-compatible anion;
b) materials having Formula (2) below

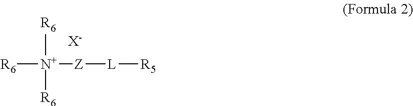

wherein
(i) R₅ is a C₅-C₂₃ hydrocarbon;
(ii) each R₆ is independently selected from the group consisting of C₁-C₄ hydrocarbon, C₁-C₄ hydroxy substituted hydrocarbon, benzyl, —(C₂H₄O)_{y}H where y is an integer from 1 to 10;
(iii) L is selected from the group consisting of —C(O)O—, —O—(O)C—,
(iv) Z is selected from the group consisting of —(CH₂)ₙ, —CH₂C(CH₃)H— where each n is independently an integer from 1 to 4;
(v) X⁻ is a softener-compatible anion;
c) materials having Formula (3) below

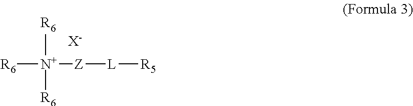

wherein
(i) R₅ is a C₅-C₂₃ hydrocarbon;
(ii) each R₆ is independently selected from the group consisting of C₁-C₄ hydrocarbon, C₁-C₄ hydroxy substituted hydrocarbon, benzyl, —(C₂H₄O)_{y}H where y is an integer from 1 to 10;
(iii) L is selected from the group consisting of —C(O)O—, —O—(O)C—,
(iv) Z is selected from the group consisting of —(CH₂)ₙ, —CH₂C(CH₃)H— where each n is independently an integer from 1 to 4;
(v) X⁻ is an anionic surfactant comprising a C₆-C₂₄ hydrocarbon.
d) materials having Formula (4) below

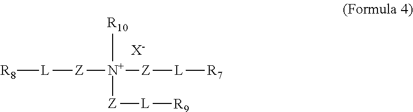

wherein:
(i) R₇, R₈ and R₉ are each independently a C₅-C₂₃ hydrocarbon;
(i) R₁₀ is selected from the group consisting of C₁-C₄ hydrocarbon, C₁-C₄ hydroxy substituted hydrocarbon, benzyl, —(C₂H₄O)_{y}H where y is an integer from 1 to 10;
(ii) L is selected from the group consisting of —C(O)O—, —O—(O)C—;
(iii) Z is selected from the group consisting of —(CH₂)ₙ, —CH₂C(CH₃)H— where each n is independently an integer from 1 to 4;
(iv) X⁻ is a softener-compatible anion;

In one aspect, said di-tail fabric softener active, mono-tail fabric softener active and ion pair fabric softener actives are selected from the group consisting of:
a) materials having Formula (1) below

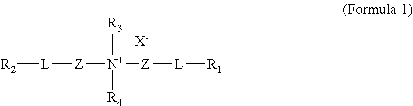

wherein:
(i) R₁ and R₂ are each independently a C₁₁-C₁₇ hydrocarbon;
(ii) R₃ and R₄ are each independently selected from the group consisting of C₁-C₂ hydrocarbon, C₁-C₂ hydroxy substituted hydrocarbon;
(iii) Z is selected from the group consisting of —(CH₂)ₙ, —CH₂C(CH₃)H— where each n is independently an integer from 1 to 2;
(iv) L is selected from the group consisting of —C(O)O—, —O—(O)C—;
(v) X− is a softener-compatible anion, selected from the group consisting of halides, sulfonates, sulfates, and nitrates.
b) materials having Formula (2) below

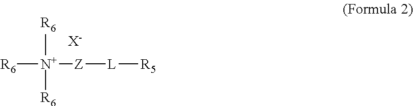

wherein
(i) R₅ is a C₁₁-C₁₇ hydrocarbon;
(ii) each R₆ is independently selected from the group consisting of C₁-C₂ hydrocarbon, C₁-C₂ hydroxy substituted hydrocarbon;
(iii) Z is selected from the group consisting of —(CH₂)ₙ, —CH₂C(CH₃)H— where each n is independently an integer from 1 to 4;
(iv) L is selected from the group consisting of —C(O)O—, —O—(O)C—;
(v) X⁻ is a softener-compatible anion, selected from the group consisting of halides, sulfonates, sulfates, and nitrates;
c) materials having Formula (3) below

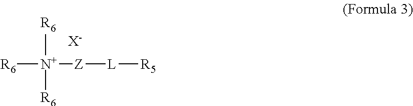

wherein
(i) R₅ is a C₁₁-C₁₇ hydrocarbon;
(ii) each R₆ is independently selected from the group consisting of C₁-C₄ hydrocarbon, C₁-C₄ hydroxy substituted hydrocarbon, benzyl, —(C₂H₄O)_{y}H where y is an integer from 1 to 10;
(iii) L is selected from the group consisting of —C(O)O—, —O—(O)C—;
(iv) Z is selected from the group consisting of —(CH₂)ₙ, —CH₂C(CH₃)H— where each n is independently an integer from 1 to 4;
(v) X− is an anionic surfactant comprising a C₆-C₂₄ hydrocarbon.

In one aspect, said di-tail fabric softener active, mono-tail fabric softener active and ion pair fabric softener actives are selected from the group consisting of:
b) materials having Formula (1) below

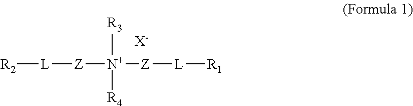

wherein:
(i) R₁ and R₂ are each independently a C₁₁-C₁₇ hydrocarbon;
(ii) R₃ and R₄ are each independently selected from the group consisting of C₁-C₂ hydrocarbon, C₁-C₂ hydroxy substituted hydrocarbon;
(iii) Z is selected from the group consisting of —(CH₂)ₙ, —CH₂C(CH₃)H— where each n is independently an integer from 1 to 2;
(iv) L is selected from the group consisting of —C(O)O—, —O—(O)C—;
(v) X⁻ is a softener-compatible anion, selected from the group consisting of chloride, bromide, methylsulfate, ethylsulfate, and methyl sulfonate.
b) materials having Formula (2) below

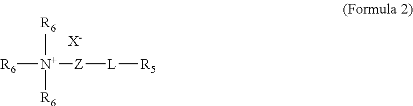

wherein
(i) R₅ is a C₁₁-C₁₇ hydrocarbon;
(ii) each R₆ is independently selected from the group consisting of C₁-C₂ hydrocarbon, C₁-C₂ hydroxy substituted hydrocarbon;
(iii) Z is selected from the group consisting of —(CH₂)ₙ, —CH₂C(CH₃)H— where each n is independently an integer from 1 to 4;
(iv) L is selected from the group consisting of —C(O)O—, —O—(O)C—;
(v) X− is a softener-compatible anion, selected from the group consisting of chloride, bromide, methylsulfate, ethylsulfate, and methyl sulfonate or anionic surfactant comprising a C₆-C₁₈ hydrocarbon
c) materials having Formula (3) below

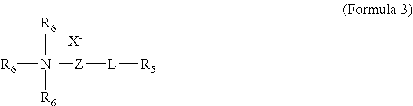

wherein
(i) R₅ is a C₁₁-C₁₇ hydrocarbon;
(ii) each R₆ is independently selected from the group consisting of C₁-C₂ hydrocarbon, C₁-C₂ hydroxy substituted hydrocarbon;
(iii) Z is selected from the group consisting of —(CH₂)ₙ, —CH₂C(CH₃)H— where each n is independently an integer from 1 to 4;
(iv) L is selected from the group consisting of —C(O)O—, —O—(O)C—;
(v) X− is a softener-compatible anion, selected from the group consisting of chloride, bromide, methylsulfate, ethylsulfate, and methyl sulfonate or anionic surfactant comprising a C₆-C₁₈ hydrocarbon.

In one aspect, for Formula 2, X− is a C₆-C₂₄ hydrocarbon that is an anionic surfactant.

In one aspect, said fabric care active comprises a fabric softening active selected from the group consisting of N,N-di(hydrogenated tallowoyloxyethyl)-N,N-dimethylammonium chloride; N,N-di(tallowoyloxyethyl)-N,N-dimethylammonium chloride; N,N-di(hydrogenated tallowoyloxyisopropyl)-N,N-dimethylammonium chloride; N,N-di(tallowoyloxyisopropyl)-N,N-dimethylammonium chloride; N,N-di(stearoyloxyisopropyl)-N,N-dimethylammonium chloride; N,N-di(palmoyloxyisopropyl)-N,N-dimethylammonium chloride; bis-(2-hydroxypropyl)-dimethylammonium chloride stearic acid diester; partially hydrogenated bis-(2-hydroxypropyl)-dimethylammonium chloride palmitic acid diester; and mixtures thereof.

In the cationic nitrogenous salts herein, the anion A⁻, which is any softener compatible anion, provides electrical neutrality. Most often, the anion used to provide electrical neutrality in these salts is from a strong acid, especially a halide, such as chloride, bromide, or iodide. However, other anions can be used, such as methylsulfate, ethylsulfate, acetate, formate, sulfate, carbonate, and the like. Chloride and methylsulfate are preferred herein as anion A. The anion can also, but less preferably, carry a double charge in which case A⁻ represents half a group.

### Step (iv)

In step (iv) the fabric is dried. This can be by any conventional drying means such as air-drying or mechanically drying in a conventional laundry drying machine.

### Step (v)

The method of the present invention comprises a step (v) of contacting the fabric from step (iv) with a soil. By ‘soil’ we mean herein any organic or inorganic material that is deposited onto the fabric that the consumer perceives as dirtying the fabric. The soil could be a stain, for example a greasy or oily food stain, or body soils such as sweat or blood. Other common stains include red food stains, clay-based stains and grass stains. Alternatively, the soil could be atmospheric soil such as chemical pollutants, dust or soot. The soil may be water-soluble or water-insoluble. These are non-limiting examples. Those skilled in the art would know what is meant by ‘soil’ in the context of the present invention. Contact of the fabric with a soil takes place in the normal use of the fabric article, for example by wearing fabric garments or typical consumer use of other fabric articles.

### Step (vi)

In step (vi), the fabric from step (v) is contacted with an aqueous wash liquor of a detergent composition wherein the detergent composition comprises a surfactant, the wash liquor comprising from 0.05 to 4 g/l of a surfactant. Step (vi) is independent from, but as described in step (i) above.

The fabric may be contacted with the respective liquor in any of steps (i), (ii), (iii) and/or (vi) at a temperature of 60° C. or less, or even 40° C. or less. In particular, the fabric may be contacted with the respective wash liquors of steps (i) and/or (vi) at a temperature of between 5° C. and 50° C., preferably between 10° C. and 30° C. The fabric may be contacted at these temperatures in the wash cycle of a domestic washing machine. In particular the present invention enables good stain/soil removal in step (vi) even at low wash temperatures such as between 10 and 30° C.

The fabric may be contacted with a laundry detergent composition in step (i) and/or step (vi) in a wash cycle of an automatic washing machine and the length of the wash cycle may be at least 30 seconds, or even at least 3 mins, or even at least 6 mins, but no more than 30 mins, or even no more than 45 mins, or even no more than 1 hour.

Other Ingredients

The laundry detergent composition of step (iii) may comprise further laundry detergent ingredients. The laundry detergent composition of step (iii) may comprise a hueing agent, a polymer or a combination thereof. Suitable detergent ingredients include: hueing agent; detersive surfactants including anionic detersive surfactants, non-ionic detersive surfactants, cationic detersive surfactants, zwitterionic detersive surfactants, amphoteric detersive surfactants, and any combination thereof; polymers including carboxylate polymers, polyethylene glycol polymers, polyester soil release polymers such as terephthalate polymers, amine polymers, cellulosic polymers, dye transfer inhibition polymers, dye lock polymers such as a condensation oligomer produced by condensation of imidazole and epichlorhydrin, optionally in ratio of 1:4:1, hexamethylenediamine derivative polymers, and any combination thereof; builders including zeolites, phosphates, citrate, and any combination thereof; buffers and alkalinity sources including carbonate salts and/or silicate salts; fillers including sulphate salts and bio-filler materials; bleach including bleach activators, sources of available oxygen, pre-formed peracids, bleach catalysts, reducing bleach, and any combination thereof; chelants; photobleach; hueing agents; brighteners; enzymes including proteases, amylases, cellulases, lipases, xylogucanases, pectate lyases, mannanases, bleaching enzymes, cutinases, and any combination thereof; fabric softeners including clay, silicones, quaternary ammonium fabric-softening agents, and any combination thereof; flocculants such as polyethylene oxide; perfume including starch encapsulated perfume accords, perfume microcapsules, perfume loaded zeolites, schif base reaction products of ketone perfume raw materials and polyamines, blooming perfumes, and any combination thereof; aesthetics including soap rings, lamellar aesthetic particles, geltin beads, carbonate and/or sulphate salt speckles, coloured clay, and any combination thereof: and any combination thereof.

Fabric Hueing Agents—

The composition may comprise a fabric hueing agent (sometimes referred to as shading, bluing or whitening agents). Typically the hueing agent provides a blue or violet shade to fabric. Hueing agents can be used either alone or in combination to create a specific shade of hueing and/or to shade different fabric types. This may be provided for example by mixing a red and green-blue dye to yield a blue or violet shade. Hueing agents may be selected from any known chemical class of dye, including but not limited to acridine, anthraquinone (including polycyclic quinones), azine, azo (e.g., monoazo, disazo, trisazo, tetrakisazo, polyazo), including premetallized azo, benzodifurane and benzodifuranone, carotenoid, coumarin, cyanine, diazahemicyanine, diphenylmethane, formazan, hemicyanine, indigoids, methane, naphthalimides, naphthoquinone, nitro and nitroso, oxazine, phthalocyanine, pyrazoles, stilbene, styryl, triarylmethane, triphenylmethane, xanthenes and mixtures thereof. Suitable fabric hueing agents include dyes, dye-clay conjugates, and organic and inorganic pigments. Suitable dyes include small molecule dyes and polymeric dyes. Suitable small molecule dyes include small molecule dyes selected from the group consisting of dyes falling into the Colour Index (C.I.) classifications of Acid, Direct, Basic, Reactive or hydrolysed Reactive, Solvent or Disperse dyes for example that are classified as Blue, Violet, Red, Green or Black, and provide the desired shade either alone or in combination. In another aspect, suitable small molecule dyes include small molecule dyes selected from the group consisting of Colour Index (Society of Dyers and Colourists, Bradford, UK) numbers Direct Violet dyes such as 9, 35, 48, 51, 66, and 99, Direct Blue dyes such as 1, 71, 80 and 279, Acid Red dyes such as 17, 73, 52, 88 and 150, Acid Violet dyes such as 15, 17, 24, 43, 49 and 50, Acid Blue dyes such as 15, 17, 25, 29, 40, 45, 75, 80, 83, 90 and 113, Acid Black dyes such as 1, Basic Violet dyes such as 1, 3, 4, 10 and 35, Basic Blue dyes such as 3, 16, 22, 47, 66, 75 and 159, Disperse or Solvent dyes such as those described in US 2008/034511 A1 or U.S. Pat. No. 8,268,016 B2, or dyes as disclosed in U.S. Pat. No. 7,208,459 B2, and mixtures thereof. In another aspect, suitable small molecule dyes include small molecule dyes selected from the group consisting of C. I. numbers Acid Violet 17, Direct Blue 71, Direct Violet 51, Direct Blue 1, Acid Red 88, Acid Red 150, Acid Blue 29, Acid Blue 113 or mixtures thereof.

Preferred dyes include dye polymers, wherein a dye group is bound to a polymeric group, optionally via a linking group. Suitable polymeric groups include (1) alkoxylated polyethyleneimine (for example as disclosed in WO2012119859), (2) polyvinyl alcohol (for example as disclosed in WO2012130492), or (3) diamine derivative of an alkylene oxide capped polyethylene glycol (for example as disclosed in WO2012126665, especially FIG. 24), or polyalkoxylated alcohol, for example as described in WO2011/011799, WO2012/054058, WO2012/166699 or WO2012/166768. One preferred class of dye polymers is obtainable by reacting a blue or violet dye containing an NH2 group with a polymer to form a covalent bond via the reacted NH2 group of the blue or violet dye and the dye polymer has an average of from 0 to 30, preferably 2 to 20, most preferably 2 to 15 repeating same units. In a preferred embodiment the monomeric units are selected from alkylene oxides, preferably ethylene oxides. Typically dye polymers will be in the form of a mixture of dye polymers in which there is a mixture of molecules having a distribution of number of monomer groups in the polymer chains, such as the mixture directly produced by the appropriate organic synthesis route, for example in the case of alkylene oxide polymers, the result of an alkoxylation reaction. Such dye polymers are typically blue or violet in colour, to give to the cloth a hue angle of 230 to 345, more preferably 250 to 330, most preferably 270 to 300. In the synthesis of dye polymers unbound blue or violet organic dyes may be present in a mixture with the final dye-polymer product. The chromophore of the blue or violet dye is preferably selected from the group consisting of: azo; anthraquinone; phthalocyanine; triphendioxazine; and, triphenylmethane. In one aspect the dye polymer is obtainable by reacting a dye containing an NH[2] group with a polymer or suitable monomer that forms a polymer in situ. Preferably the NH[2] is covalently bound to an aromatic ring of the dye. Unbound dye is formed when the dye does not react with polymer. Preferred dyes containing —NH[2] groups for such reactions are selected from: acid violet 1; acid violet 3; acid violet 6; acid violet 1 1; acid violet 13; acid violet 14; acid violet 19; acid violet 20; acid violet 36; acid violet 36:1; acid violet 41; acid violet 42; acid violet 43; acid violet 50; acid violet 51; acid violet 63; acid violet 48; acid blue 25; acid blue 40; acid blue 40:1; acid blue 41; acid blue 45; acid blue 47; acid blue 49; acid blue 51; acid blue 53; acid blue 56; acid blue 61; acid blue 61:1; acid blue 62; acid blue 69; acid blue 78; acid blue 81:1; acid blue 92; acid blue 96; acid blue 108; acid blue 1 1 1; acid blue 215; acid blue 230; acid blue 277; acid blue 344; acid blue 1 17; acid blue 124; acid blue 129; acid blue 129:1; acid blue 138; acid blue 145; direct violet 99; direct violet 5; direct violet 72; direct violet 16; direct violet 78; direct violet 77; direct violet 83; food black 2; direct blue 33; direct blue 41; direct blue 22; direct blue 71; direct blue 72; direct blue 74; direct blue 75; direct blue 82; direct blue 96; direct blue 1 10; direct blue 1 1 1; direct blue 120; direct blue 120:1; direct blue 121; direct blue 122; direct blue 123; direct blue 124; direct blue 126; direct blue 127; direct blue 128; direct blue 129; direct blue 130; direct blue 132; direct blue 133; direct blue 135; direct blue 138; direct blue 140; direct blue 145; direct blue 148; direct blue 149; direct blue 159; direct blue 162; direct blue 163; food black 2; food black 1 wherein the acid amide group is replaced by NH[2]; Basic Violet 2; Basic Violet 5; Basic Violet 12; Basic Violet 14; Basic Violet 8; Basic Blue 12; Basic Blue 16; Basic Blue 17; Basic Blue 47; Basic Blue 99; disperse blue 1; disperse blue 5; disperse blue 6; disperse blue 9; disperse blue 1 1; disperse blue 19; disperse blue 20; disperse blue 28; disperse blue 40; disperse blue 56; disperse blue 60; disperse blue 81; disperse blue 83; disperse blue 87; disperse blue 104; disperse blue 1 18; disperse violet 1; disperse violet 4, disperse violet 8, disperse violet 17, disperse violet 26; disperse violet 28; solvent violet 26; solvent blue 12; solvent blue 13; solvent blue 18; solvent blue 68. Further preferred dyes are selected from mono-azo dyes which contain a phenyl group directly attached to the azo group, wherein the phenyl group has an NH[2] groups covalent bound to it. For example a mono-azo thiophene dye. The polymer chain may be selected from polyalkylene oxides. The polymer chain and/or the dye chromophore group may optionally carry anionic or cationic groups. Examples of polyoxyalkylene oxide chains include ethylene oxide, propylene oxide, glycidol oxide, butylene oxide and mixtures thereof.

Suitable polymeric dyes include polymeric dyes selected from the group consisting of polymers containing covalently bound (sometimes referred to as conjugated) chromogens, (dye-polymer conjugates), for example polymers with chromogens co-polymerized into the backbone of the polymer and mixtures thereof. Polymeric dyes include those described in WO2011/98355, US 2012/225803 A1, US 2012/090102 A1, U.S. Pat. No. 7,686,892 B2, and WO2010/142503.

In another aspect, suitable polymeric dyes include polymeric dyes selected from the group consisting of fabric-substantive colorants sold under the name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) covalently bound to a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colourants, alkoxylated thiophene polymeric colourants, and mixtures thereof.

Preferred hueing dyes include the whitening agents found in WO 08/87497 A1, WO2011/011799 and US 2012/129752 A1. Preferred hueing agents for use in the present invention may be the preferred dyes disclosed in these references, including those selected from Examples 1-42 in Table 5 of WO2011/011799. Other preferred dyes are disclosed in U.S. Pat. No. 8,138,222B2, especially claim 1 of U.S. Pat. No. 8,138,222B2. Other preferred dyes are disclosed in U.S. Pat. No. 7,909,890 B2.

Suitable dye clay conjugates include dye clay conjugates selected from the group comprising at least one cationic/basic dye and a smectite clay, and mixtures thereof. In another aspect, suitable dye clay conjugates include dye clay conjugates selected from the group consisting of one cationic/basic dye selected from the group consisting of C.I. Basic Yellow 1 through 108, C.I. Basic Orange 1 through 69, C.I. Basic Red 1 through 118, C.I. Basic Violet 1 through 51, C.I. Basic Blue 1 through 164, C.I. Basic Green 1 through 14, C.I. Basic Brown 1 through 23, CI Basic Black 1 through 11, and a clay selected from the group consisting of Montmorillonite clay, Hectorite clay, Saponite clay and mixtures thereof. In still another aspect, suitable dye clay conjugates include dye clay conjugates selected from the group consisting of: Montmorillonite Basic Blue B7 C.I. 42595 conjugate, Montmorillonite Basic Blue B9 C.I. 52015 conjugate, Montmorillonite Basic Violet V3 C.I. 42555 conjugate, Montmorillonite Basic Green G1 C.I. 42040 conjugate, Montmorillonite Basic Red R1 C.I. 45160 conjugate, Montmorillonite C.I. Basic Black 2 conjugate, Hectorite Basic Blue B7 C.I. 42595 conjugate, Hectorite Basic Blue B9 C.I. 52015 conjugate, Hectorite Basic Violet V3 C.I. 42555 conjugate, Hectorite Basic Green G1 C.I. 42040 conjugate, Hectorite Basic Red R1 C.I. 45160 conjugate, Hectorite C.I. Basic Black 2 conjugate, Saponite Basic Blue B7 C.I. 42595 conjugate, Saponite Basic Blue B9 C.I. 52015 conjugate, Saponite Basic Violet V3 C.I. 42555 conjugate, Saponite Basic Green G1 C.I. 42040 conjugate, Saponite Basic Red R1 C.I. 45160 conjugate, Saponite C.I. Basic Black 2 conjugate and mixtures thereof.

Suitable pigments include pigments selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C1-C3-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule and mixtures thereof.

In another aspect, suitable pigments include pigments selected from the group consisting of Ultramarine Blue (C.I. Pigment Blue 29), Ultramarine Violet (C.I. Pigment Violet 15) and mixtures thereof.

The aforementioned fabric hueing agents can be used in combination (any mixture of fabric hueing agents can be used).

Polymer: Suitable polymers include carboxylate polymers, polyethylene glycol polymers, polyester soil release polymers such as terephthalate polymers, amine polymers, cellulosic polymers, dye transfer inhibition polymers, dye lock polymers such as a condensation oligomer produced by condensation of imidazole and epichlorhydrin, optionally in ratio of 1:4:1, hexamethylenediamine derivative polymers, and any combination thereof.

Carboxylate polymer: Suitable carboxylate polymers include maleate/acrylate random copolymer or polyacrylate homopolymer. The carboxylate polymer may be a polyacrylate homopolymer having a molecular weight of from 4,000 Da to 9,000 Da, or from 6,000 Da to 9,000 Da. Other suitable carboxylate polymers are co-polymers of maleic acid and acrylic acid, and may have a molecular weight in the range of from 4,000 Da to 90,000 Da.

Other suitable carboxylate polymers are co-polymers comprising: (i) from 50 to less than 98 wt % structural units derived from one or more monomers comprising carboxyl groups; (ii) from 1 to less than 49 wt % structural units derived from one or more monomers comprising sulfonate moieties; and (iii) from 1 to 49 wt % structural units derived from one or more types of monomers selected from ether bond-containing monomers represented by formulas (I) and (II):

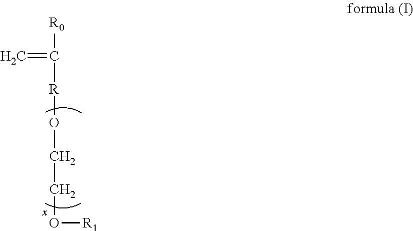

wherein in formula (I), R₀ represents a hydrogen atom or CH₃ group, R represents a CH₂ group, CH₂CH₂ group or single bond, X represents a number 0-5 provided X represents a number 1-5 when R is a single bond, and R₁ is a hydrogen atom or C₁ to C₂₀ organic group;

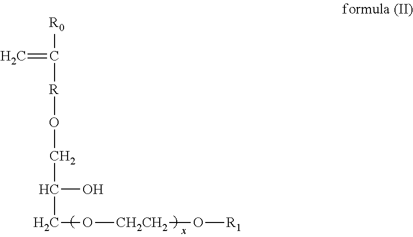

in formula (II), R₀ represents a hydrogen atom or CH₃ group, R represents a CH₂ group, CH₂CH₂ group or single bond, X represents a number 0-5, and R₁ is a hydrogen atom or C₁ to C₂₀ organic group.

Polyethylene glycol polymer: Suitable polyethylene glycol polymers include random graft co-polymers comprising: (i) hydrophilic backbone comprising polyethylene glycol; and (ii) hydrophobic side chain(s) selected from the group consisting of: C₄-C₂₅ alkyl group, polypropylene, polybutylene, vinyl ester of a saturated C₁-C₆ mono-carboxylic acid, C₁-C₆ alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22.

Polyester soil release polymers: Suitable polyester soil release polymers have a structure as defined by one of the following structures (I), (II) or (III):

—[(OCHR¹—CHR²)ₐ—O—OC—Ar—CO—]_{d} (I)

—[(OCHR³—CHR⁴)_{b}—O—OC-sAr—CO—]ₑ (II)

—[(OCHR⁵—CHR⁶)_{c}—OR⁷]_{f} (III)

wherein:

a, b and c are from 1 to 200;

d, e and f are from 1 to 50;

Ar is a 1,4-substituted phenylene;

sAr is 1,3-substituted phenylene substituted in position 5 with SO₃Me;

Me is H, Na, Li, K, Mg/2, Ca/2, Al/3, ammonium, mono-, di-, tri-, or tetraalkylammonium wherein the alkyl groups are C₁-C₁₈ alkyl or C₂-C₁₀ hydroxyalkyl, or any mixture thereof;

R¹, R², R³, R⁴, R⁵ and R⁶ are independently selected from H or C₁-C₁₈ n- or iso-alkyl; and

R⁷ is a linear or branched C₁-C₁₈ alkyl, or a linear or branched C₂-C₃₀ alkenyl, or a cycloalkyl group with 5 to 9 carbon atoms, or a C₈-C₃₀ aryl group, or a C₆-C₃₀ arylalkyl group.

Suitable polyester soil release polymers are terephthalate polymers having the structure of formula (I) or (II) above.

Suitable polyester soil release polymers include the Repel-o-tex series of polymers such as Repel-o-tex SF2 (Rhodia) and/or the Texcare series of polymers such as Texcare SRA300 (Clariant).

Amine polymer: Suitable amine polymers include polyethylene imine polymers, such as alkoxylated polyalkyleneimines, optionally comprising a polyethylene and/or polypropylene oxide block.

Cellulosic polymer: The composition can comprise cellulosic polymers, such as polymers selected from alkyl cellulose, alkyl alkoxyalkyl cellulose, carboxyalkyl cellulose, alkyl carboxyalkyl, and any combination thereof. Suitable cellulosic polymers are selected from carboxymethyl cellulose, methyl cellulose, methyl hydroxyethyl cellulose, methyl carboxymethyl cellulose, and mixtures thereof. The carboxymethyl cellulose can have a degree of carboxymethyl substitution from 0.5 to 0.9 and a molecular weight from 100,000 Da to 300,000 Da. Another suitable cellulosic polymer is hydrophobically modified carboxymethyl cellulose, such as Finnfix SH-1 (CP Kelco).

Other suitable cellulosic polymers may have a degree of substitution (DS) of from 0.01 to 0.99 and a degree of blockiness (DB) such that either DS+DB is of at least 1.00 or DB+2DS-DS² is at least 1.20. The substituted cellulosic polymer can have a degree of substitution (DS) of at least 0.55. The substituted cellulosic polymer can have a degree of blockiness (DB) of at least 0.35. The substituted cellulosic polymer can have a DS+DB, of from 1.05 to 2.00. A suitable substituted cellulosic polymer is carboxymethylcellulose.

Another suitable cellulosic polymer is cationically modified hydroxyethyl cellulose.

Dye transfer inhibitor (DTI) polymer: The laundry detergent compositions may comprise DTI polymers. Suitable DTIs include polyamine N-oxide polymers, copolymers of N-vinylpyrrolidone and N-vinylimidazole, polyvinylpyrrolidone polymers, polyvinyloxazolidones and polyvinylimidazoles or mixtures thereof. The DTI polymers discussed above are well known in the art and commercially available, for example PVP-K15 and K30 (Ashland), Sokalan HP165, HP50, HP53, HP59, HP56K, HP56, HP66 (BASF), Chromabond S-400, S403E and S-100 (Ashland), and Polyquart FDI (Cognis).

Builder: Suitable builders include zeolites, phosphates, citrates, and any combination thereof.

Zeolite builder: The composition may be substantially free of zeolite builder. Substantially free of zeolite builder typically means comprises from 0 wt % to 10 wt %, zeolite builder, or to 8 wt %, or to 6 wt %, or to 4 wt %, or to 3 wt %, or to 2 wt %, or even to 1 wt % zeolite builder. Substantially free of zeolite builder preferably means “no deliberately added” zeolite builder. Typical zeolite builders include zeolite A, zeolite P, zeolite MAP, zeolite X and zeolite Y.

Phosphate builder: The composition may be substantially free of phosphate builder. Substantially free of phosphate builder typically means comprises from 0 wt % to 10 wt % phosphate builder, or to 8 wt %, or to 6 wt %, or to 4 wt %, or to 3 wt %, or to 2 wt %, or even to 1 wt % phosphate builder. Substantially free of zeolite builder preferably preferably means “no deliberately added” phosphate builder. A typical phosphate builder is sodium tri-polyphosphate (STPP).

Citrate: A suitable citrate is sodium citrate. However, citric acid may also be incorporated into the composition, which can form citrate in the wash liquor.

Buffer and alkalinity source: Suitable buffers and alkalinity sources include carbonate salts and/or silicate salts and/or double salts such as burkeitte.

Carbonate salt: A suitable carbonate salt is sodium carbonate and/or sodium bicarbonate. The carbonate salt may have a weight average mean particle size of from 100 to 500 micrometers. Alternatively, the carbonate salt may have a weight average mean particle size of from 10 to 25 micrometers.

Silicate salt: The silicate can be crystalline or amorphous. Suitable crystalline silicates include crystalline layered silicate, such as SKS-6. Other suitable silicates include 1.6R silicate and/or 2.0R silicate. A suitable silicate salt is sodium silicate. Another suitable silicate salt is sodium metasilicate.

Sulphate salt: A suitable sulphate salt is sodium sulphate. The sulphate salt may have a weight average mean particle size of from 100 to 500 micrometers, alternatively, the sulphate salt may have a weight average mean particle size of from 10 to 45 micrometers.

Bleach activator: Suitable bleach activators include: tetraacetylethylenediamine (TAED); oxybenzene sulphonates such as nonanoyl oxybenzene sulphonate (NOBS), caprylamidononanoyl oxybenzene sulphonate (NACA-OBS), 3,5,5-trimethyl hexanoyloxybenzene sulphonate (Iso-NOBS), dodecyl oxybenzene sulphonate (LOBS), and any mixture thereof; caprolactams; pentaacetate glucose (PAG); nitrile quaternary ammonium; imide bleach activators, such as N-nonanoyl-N-methyl acetamide; and any mixture thereof.

Source of available oxygen: A suitable source of available oxygen (AvOx) is a source of hydrogen peroxide, such as percarbonate salts and/or perborate salts, such as sodium percarbonate. The source of peroxygen may be at least partially coated, or even completely coated, by a coating ingredient such as a carbonate salt, a sulphate salt, a silicate salt, borosilicate, or any mixture thereof, including mixed salts thereof. Suitable percarbonate salts can be prepared by a fluid bed process or by a crystallization process. Suitable perborate salts include sodium perborate mono-hydrate (PB1), sodium perborate tetra-hydrate (PB4), and anhydrous sodium perborate which is also known as fizzing sodium perborate. Other suitable sources of AvOx include persulphate, such as oxone. Another suitable source of AvOx is hydrogen peroxide.

Pre-formed peracid: A suitable pre-formed peracid is N,N-pthaloylamino peroxycaproic acid (PAP).

Bleach catalyst: Suitable bleach catalysts include oxaziridinium-based bleach catalysts, transition metal bleach catalysts and bleaching enzymes.

Oxaziridinium-based bleach catalyst: A suitable oxaziridinium-based bleach catalyst has the formula:

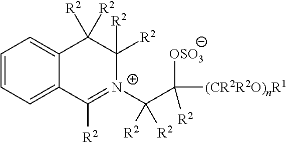

wherein: R¹ is selected from the group consisting of: H, a branched alkyl group containing from 3 to 24 carbons, and a linear alkyl group containing from 1 to 24 carbons; R¹ can be a branched alkyl group comprising from 6 to 18 carbons, or a linear alkyl group comprising from 5 to 18 carbons, R¹ can be selected from the group consisting of: 2-propylheptyl, 2-butyloctyl, 2-pentylnonyl, 2-hexyldecyl, n-hexyl, n-octyl, n-decyl, n-dodecyl, n-tetradecyl, n-hexadecyl, n-octadecyl, iso-nonyl, iso-decyl, iso-tridecyl and iso-pentadecyl; R² is independently selected from the group consisting of: H, a branched alkyl group comprising from 3 to 12 carbons, and a linear alkyl group comprising from 1 to 12 carbons; optionally R² is independently selected from H and methyl groups; and n is an integer from 0 to 1.

Transition metal bleach catalyst: The composition may include transition metal bleach catalyst, typically comprising copper, iron, titanium, ruthenium, tungsten, molybdenum, and/or manganese cations. Suitable transition metal bleach catalysts are manganese-based transition metal bleach catalysts.

Reducing bleach: The composition may comprise a reducing bleach. However, the composition may be substantially free of reducing bleach; substantially free means “no deliberately added”. Suitable reducing bleach include sodium sulphite and/or thiourea dioxide (TDO).

Co-bleach particle: The composition may comprise a co-bleach particle. Typically, the co-bleach particle comprises a bleach activator and a source of peroxide. It may be highly suitable for a large amount of bleach activator relative to the source of hydrogen peroxide to be present in the co-bleach particle. The weight ratio of bleach activator to source of hydrogen peroxide present in the co-bleach particle can be at least 0.3:1, or at least 0.6:1, or at least 0.7:1, or at least 0.8:1, or at least 0.9:1, or at least 1.0:1.0, or even at least 1.2:1 or higher.

The co-bleach particle can comprise: (i) bleach activator, such as TAED; and (ii) a source of hydrogen peroxide, such as sodium percarbonate. The bleach activator may at least partially, or even completely, enclose the source of hydrogen peroxide.

The co-bleach particle may comprise a binder. Suitable binders are carboxylate polymers such as polyacrylate polymers, and/or surfactants including non-ionic detersive surfactants and/or anionic detersive surfactants such as linear C₁₁-C₁₃ alkyl benzene sulphonate.

The co-bleach particle may comprise bleach catalyst, such as an oxaziridium-based bleach catalyst.

Chelant: Suitable chelants are selected from: diethylene triamine pentaacetate, diethylene triamine penta(methyl phosphonic acid), ethylene diamine-N′N′-disuccinic acid, ethylene diamine tetraacetate, ethylene diamine tetra(methylene phosphonic acid), hydroxyethane di(methylene phosphonic acid), and any combination thereof. A suitable chelant is ethylene diamine-N′N′-disuccinic acid (EDDS) and/or hydroxyethane diphosphonic acid (HEDP). The laundry detergent composition may comprise ethylene diamine-N′N′-disuccinic acid or salt thereof. The ethylene diamine-N′N′-disuccinic acid may be in S,S enantiomeric form. The composition may comprise 4,5-dihydroxy-m-benzenedisulfonic acid disodium salt. Suitable chelants may also be calcium crystal growth inhibitors.

Calcium carbonate crystal growth inhibitor: The composition may comprise a calcium carbonate crystal growth inhibitor, such as one selected from the group consisting of: 1-hydroxyethanediphosphonic acid (HEDP) and salts thereof; N,N-dicarboxymethyl-2-aminopentane-1,5-dioic acid and salts thereof; 2-phosphonobutane-1,2,4-tricarboxylic acid and salts thereof; and any combination thereof.

Photobleach: Suitable photobleaches are zinc and/or aluminium sulphonated phthalocyanines.

Brightener: The laundry detergent composition may comprise fluorescent brightener. Preferred classes of fluorescent brightener are: Di-styryl biphenyl compounds, e.g. Tinopal™ CBS-X, Di-amino stilbene di-sulfonic acid compounds, e.g. Tinopal™ DMS pure Xtra and Blankophor™ HRH, and Pyrazoline compounds, e.g. Blankophor™ SN. Preferred fluorescers are: sodium 2 (4-styryl-3-sulfophenyl)-2H-napthol[1,2-d]triazole, disodium 4,4′-bis{[(4-anilino-6-(N methyl-N-2 hydroxyethyl)amino 1,3,5-triazin-2-yl)]; amino}stilbene-2-2′ disulfonate, disodium 4,4′-bis{[(4-anilino-6-morpholino-1,3,5-triazin-2-yl)]amino}stilbene-2-2′ disulfonate, and disodium 4,4′-bis(2-sulfostyryl)biphenyl.

A particularly preferred fluorescent brightener is C.I. Fluorescent Brightener 260 having the following structure. For solid detergent compositions, this brightener may be used in its beta or alpha crystalline forms, or a mixture of these forms.

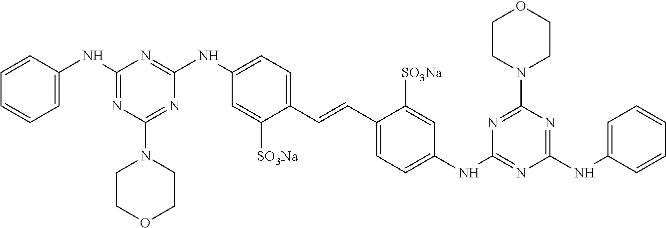

Enzyme: Suitable enzymes include proteases, amylases, cellulases, lipases, xylogucanases, pectate lyases, mannanases, bleaching enzymes, cutinases, and mixtures thereof.

For the enzymes, accession numbers and IDs shown in parentheses refer to the entry numbers in the databases Genbank, EMBL and/or Swiss-Prot. For any mutations, standard 1-letter amino acid codes are used with a * representing a deletion. Accession numbers prefixed with DSM refer to micro-organisms deposited at Deutsche Sammlung von Mikroorganismen and Zellkulturen GmbH, Mascheroder Weg 1b, 38124 Brunswick (DSMZ).

Protease: The composition may comprise a protease. Suitable proteases include metalloproteases and/or serine proteases, including neutral or alkaline microbial serine proteases, such as subtilisins (EC 3.4.21.62). Suitable proteases include those of animal, vegetable or microbial origin. In one aspect, such suitable protease may be of microbial origin. The suitable proteases include chemically or genetically modified mutants of the aforementioned suitable proteases. In one aspect, the suitable protease may be a serine protease, such as an alkaline microbial protease or/and a trypsin-type protease. Examples of suitable neutral or alkaline proteases include:

(a) subtilisins (EC 3.4.21.62), including those derived from Bacillus, such as Bacillus lentus, Bacillus alkalophilus (P27963, ELYA_BACAO), Bacillus subtilis, Bacillus amyloliquefaciens (P00782, SUBT_BACAM), Bacillus pumilus (P07518) and Bacillus gibsonii (DSM14391).

(b) trypsin-type or chymotrypsin-type proteases, such as trypsin (e.g. of porcine or bovine origin), including the Fusarium protease and the chymotrypsin proteases derived from Cellumonas (A2RQE2).

(c) metalloproteases, including those derived from Bacillus amyloliquefaciens (P06832, NPRE BACAM).

Suitable proteases include those derived from Bacillus gibsonii or Bacillus Lentus such as subtilisin 309 (P29600) and/or DSM 5483 (P29599).

Suitable commercially available protease enzymes include: those sold under the trade names Alcalase®, Savinase®, Primase®, Durazym®, Polarzyme®, Kannase®, Liquanase®, Liquanase Ultra®, Savinase Ultra®, Ovozyme®, Neutrase®, Everlase® and Esperase® by Novozymes A/S (Denmark); those sold under the tradename Maxatase®, Maxacal®, Maxapem®, Properase®, Purafect®, Purafect Prime®, Purafect Ox®, FN3®, FN4®, Excellase® and Purafect OXP® by Genencor International; those sold under the tradename Opticlean® and Optimase® by Solvay Enzymes; those available from Henkel/Kemira, namely BLAP (P29599 having the following mutations S99D+S101R+S103A+V104I+G159S), and variants thereof including BLAP R (BLAP with S3T+V4I+V199M+V205I+L217D), BLAP X (BLAP with S3T+V4I+V205I) and BLAP F49 (BLAP with S3T+V4I+A194P+V199M+V205I+L217D) all from Henkel/Kemira; and KAP (Bacillus alkalophilus subtilisin with mutations A230V+S256G+S259N) from Kao.

Other suitable protease enzymes are fungal serine proteases. Suitable enzymes are variants or wild-types of the fungal serine proteases endogenous to Trichoderma reesei strain QM9414, Malbranchea cinnamomea strain ALK04122, Fusarium graminearum strain ALK01726, Fusarium equiseti strain CBS 119568 and Fusarium acuminatum strain CBS 124084. Examples of commercially available fungal serine proteases are Biotouch ROC and Biotouch Novia, both supplied by AB Enzymes, Darmstadt, Germany.

Amylase: Suitable amylases are alpha-amylases, including those of bacterial or fungal origin. Chemically or genetically modified mutants (variants) are included. A suitable alkaline alpha-amylase is derived from a strain of Bacillus, such as Bacillus licheniformis, Bacillus amyloliquefaciens, Bacillus stearothermophilus, Bacillus subtilis, or other Bacillus sp., such as Bacillus sp. NCIB 12289, NCIB 12512, NCIB 12513, sp 707, DSM 9375, DSM 12368, DSMZ no. 12649, KSM AP1378, KSM K36 or KSM K38. Suitable amylases include:

(a) alpha-amylase derived from Bacillus licheniformis (P06278, AMY_BACLI), and variants thereof, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

(b) AA560 amylase (CBU30457, HD066534) and variants thereof, especially the variants with one or more substitutions in the following positions: 26, 30, 33, 82, 37, 106, 118, 128, 133, 149, 150, 160, 178, 182, 186, 193, 203, 214, 231, 256, 257, 258, 269, 270, 272, 283, 295, 296, 298, 299, 303, 304, 305, 311, 314, 315, 318, 319, 339, 345, 361, 378, 383, 419, 421, 437, 441, 444, 445, 446, 447, 450, 461, 471, 482, 484, optionally that also contain the deletions of D183* and G184*.

(c) variants exhibiting at least 90% identity with the wild-type enzyme from Bacillus SP722 (CBU30453, HD066526), especially variants with deletions in the 183 and 184 positions.

Suitable commercially available alpha-amylases are Duramyl®, Liquezyme® Termamyl®, Termamyl Ultra®, Natalase®, Supramyl®, Stainzyme®, Stainzyme Plus®, Fungamyl® and BAN® (Novozymes A/S), Bioamylase® and variants thereof (Biocon India Ltd.), Kemzym® AT 9000 (Biozym Ges. m.b.H, Austria), Rapidase®, Purastar®, Optisize HT Plus®, Enzysize®, Powerase® and Purastar Oxam®, Maxamyl® (Genencor International Inc.) and KAM® (KAO, Japan). Suitable amylases are Natalase®, Stainzyme® and Stainzyme Plus®.

Cellulase: The composition may comprise a cellulase. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum.

Commercially available cellulases include Celluzyme®, and Carezyme® (Novozymes A/S), Clazinase®, and Puradax HA® (Genencor International Inc.), and KAC-500(B)® (Kao Corporation).

The cellulase can include microbial-derived endoglucanases exhibiting endo-beta-1,4-glucanase activity (E.C. 3.2.1.4), including a bacterial polypeptide endogenous to a member of the genus Bacillus sp. AA349 and mixtures thereof. Suitable endoglucanases are sold under the tradenames Celluclean® and Whitezyme® (Novozymes A/S, Bagsvaerd, Denmark).

The composition may comprise a cleaning cellulase belonging to Glycosyl Hydrolase family 45 having a molecular weight of from 17 kDa to 30 kDa, for example the endoglucanases sold under the tradename Biotouch® NCD, DCC and DCL (AB Enzymes, Darmstadt, Germany).

Suitable cellulases may also exhibit xyloglucanase activity, such as Whitezyme®. Xyloglucanase: Suitable xyloglucanase enzymes may have enzymatic activity towards both xyloglucan and amorphous cellulose substrates. The enzyme may be a glycosyl hydrolase (GH) selected from GH families 5, 12, 44 or 74. The glycosyl hydrolase selected from GH family 44 is particularly suitable. Suitable glycosyl hydrolases from GH family 44 are the XYG1006 glycosyl hydrolase from Paenibacillus polyxyma (ATCC 832) and variants thereof.

Pectate lyase: Suitable pectate lyases are either wild-types or variants of Bacillus-derived pectate lyases (CAF05441, AAU25568) sold under the tradenames Pectawash®, Pectaway® and X-Pect® (from Novozymes A/S, Bagsvaerd, Denmark).

Mannanase: Suitable mannanases are sold under the tradenames Mannaway® (from Novozymes A/S, Bagsvaerd, Denmark), and Purabrite® (Genencor International Inc., Palo Alto, Calif.).

Bleaching enzyme: Suitable bleach enzymes include oxidoreductases, for example oxidases such as glucose, choline or carbohydrate oxidases, oxygenases, catalases, peroxidases, like halo-, chloro-, bromo-, lignin-, glucose- or manganese-peroxidases, dioxygenases or laccases (phenoloxidases, polyphenoloxidases). Suitable commercial products are sold under the Guardzyme® and Denilite® ranges from Novozymes. It may be advantageous for additional organic compounds, especially aromatic compounds, to be incorporated with the bleaching enzyme; these compounds interact with the bleaching enzyme to enhance the activity of the oxidoreductase (enhancer) or to facilitate the electron flow (mediator) between the oxidizing enzyme and the stain typically over strongly different redox potentials.

Other suitable bleaching enzymes include perhydrolases, which catalyse the formation of peracids from an ester substrate and peroxygen source. Suitable perhydrolases include variants of the Mycobacterium smegmatis perhydrolase, variants of so-called CE-7 perhydrolases, and variants of wild-type subtilisin Carlsberg possessing perhydrolase activity.

Identity: The relativity between two amino acid sequences is described by the parameter “identity”. For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

Fabric-softener: Suitable fabric-softening agents include clay, silicone and/or quaternary ammonium compounds. Suitable clays include montmorillonite clay, hectorite clay and/or laponite clay. A suitable clay is montmorillonite clay. Suitable silicones include amino-silicones and/or polydimethylsiloxane (PDMS). A suitable fabric softener is a particle comprising clay and silicone, such as a particle comprising montmorillonite clay and PDMS.

Flocculant: Suitable flocculants include polyethylene oxide; for example having an average molecular weight of from 300,000 Da to 900,000 Da.

Suds suppressor: Suitable suds suppressors include silicone and/or fatty acid such as stearic acid.

Perfume: Suitable perfumes include perfume microcapsules, polymer assisted perfume delivery systems including Schiff base perfume/polymer complexes, starch-encapsulated perfume accords, perfume-loaded zeolites, blooming perfume accords, and any combination thereof. A suitable perfume microcapsule is melamine formaldehyde based, typically comprising perfume that is encapsulated by a shell comprising melamine formaldehyde. It may be highly suitable for such perfume microcapsules to comprise cationic and/or cationic precursor material in the shell, such as polyvinyl formamide (PVF) and/or cationically modified hydroxyethyl cellulose (catHEC).

Aesthetic: Suitable aesthetic particles include soap rings, lamellar aesthetic particles, geltin beads, carbonate and/or sulphate salt speckles, coloured clay particles, and any combination thereof.

### EXAMPLES

### Example 1

The improved soil removal benefit of the method of the present invention was demonstrated in the following experiment.

A composition was prepared comprising a Western European liquid detergent detailed in table 1. This composition was labeled pre-condition composition 1.

A second pre-condition composition was prepared which was identical to pre-condition composition 1, but which was also used in conjunction with a rinse additive detailed in table 2.

A third pre-condition composition was prepared which was identical to pre-condition composition 2, but which also comprised a variant having at least 90% sequence identity to wild-type lipase from Thermomyces lanuginosus and having sequence substitutions T231R and N233R.

A fourth pre-condition composition was prepared which was identical to pre-condition composition 2, but which was also used in conjunction with a Soil Release Polymer (SRP).

A fifth pre-condition composition was prepared which was identical to pre-condition composition 4, but which also comprised a variant having at least 90% sequence identity to wild-type lipase from Thermomyces lanuginosus and having sequence substitutions T231R and N233R.

To summarise;
1. pre-condition composition only
2. pre-condition composition in conjunction with rinse additive
3. pre-condition composition comprising Lipex® in conjunction with rinse additive
4. pre-condition composition comprising SRP in conjunction with rinse additive
5. pre-condition composition comprising SRP and Lipex® in conjunction with rinse additive

Standard fabric swatches TF7436-M polycotton (25×20 cm swatches) and Dacron 64 polyester (25×20 cm swatches) were obtained from Westlairds. Two of each of these were added to a washing machine together with 2.5 kg of cotton sheets and cotton towels supplied by Test Fabrics added as additional ballast.

The swatches were then washed on the ‘short cotton cycle’ (30° C.) at 1600 rpm together with the detergent composition detailed in table 1 such that the 13 litre wash liquor comprised 2692 ppm of the detergent. A 30 g dose of the rinse additive detailed in table 2 was added to the rinse compartment in the drawer of the washing machine to be added during the rinse step of the washing machine cycle. The lipid esterase was added to the rinse compartment in the drawer of the washing machine such that the concentration of 1 ppm active enzyme protein would be delivered during the rinse step of the washing machine cycle. The soil release polymer was added at a level of 0.2% w/w to the rinse compartment in the drawer of the washing machine to be delivered during the rinse step of the washing machine cycle. The order of addition to the rinse compartment was rinse additive, then lipid esterase, then soil release polymer. After the wash cycle was complete, the fabrics were then dried on a line. This was repeated so that all swatches had been washed four times together with the same pre-condition treatment composition during the wash and rinse additive, lipid esterase and soil release polymer added through the rinse.

5×5 cm swatches of TF7436-M and Dacron 64 from each pre-condition treatment were prepared and stained with 200 μL of SV13-dyed lard (Asda lard batch 130R7, SV13%, batch SPt001013) and were stored at 32° C./80% rh overnight.

The stained swatches were then washed in a Tergotometer (0.8 L pot) in the presence of a Western European liquid detergent detailed in table 1 at a total concentration of 2692 ppm. Ballast fabric made up of knitted cotton fabric was also added, with an overall fabric load of 26.7 g. Wash conditions in the Tergotometer were 200 rpm, wash time 20 minutes at 30° C. and rinse time of five minutes. Fabrics were then air dried overnight on metal racks.

TABLE 1

| Detergent Composition | (wt %) |
|---|---|
| 1,2-Propanediol | 17.3 |
| Linear alkylbenzene sulfonate | 14.8 |
| C₁₂₋₁₄ alkyl-7-ethoxylate | 13.0 |
| C₁₂₋₁₈ fatty acid | 12.7 |
| Sodium C₁₂₋₁₄ alkyl ethoxy 3 sulfate | 11.1 |
| Monoethanolamine | 7.5 |
| Glycerol | 6.2 |
| Ethoxylated polyethylenepolyamine | 4.0 |
| 1-hydroxyethyidene-1,1-diphosphonic acid | 1.2 |
| Citric acid | 0.6 |
| Tinopal ® CBS-X B49 | 0.2 |
| Magnesium chloride | 0.2 |
| Sodium hydroxide | 0.1 |
| Water, aesthetics, (dyes, perfumes) and | Balance to |
| minors (solvents and structurants) | 100% |

TABLE 2

| Rinse Additive Composition | (wt %) |
|---|---|
| Silicone HC306 | 12.0 |
| Poly(acrylamide-methacrylamidopropyltrimethyl | 0.2 |
| ammonium chloride (PAMMAPTAC) | |
| Tallow alkyl ethoxylated alcohol with an average | 0.1 |
| degree of ethoxylation of 80. (TAE₈₀) | |
| Proxel ™ GXL | 0.02 |
| Perfume Best B | 1.0 |
| Water | 86.68 |

Lipid esterase was added to the rinse liquor at a concentration of 1 ppm (active enzyme protein) Soil release polymer Texcare® SRA300 was added at 0.2% w/w.

Stain removal was quantified using commercially available Digieye software to calculate percentage stain removal from L*a*b* values. The software generates the L value, the a value and the b value, and percentage stain removal was calculated using the following equation;

% SR (stain removal)=100*((ΔE_{b}−ΔEₐ)/ΔE_{b})

ΔE_{b}=√((L_{c}−L_{b})²+(a_{c}−a_{b})²+b_{c}−bₐ)²)

ΔEₐ=√((L_{c}−Lₐ)²+(a_{c}−aₐ)²+b_{c}−bₐ)²)

Subscript ‘b’ denotes data for the stain before washing
Subscript ‘a’ denotes data for the stain after washing
Subscript ‘c’ denotes data for the unstained fabric
Thus, L*a*b* values are taken of the unstained fabric, of the stained fabric before washing and of the stained fabric after washing.
Results can be seen in table 3

TABLE 3

| | TF7436-M | | Dacron 64 | |
|---|---|---|---|---|
| Pre-condition | | Standard | | Standard |
| composition | % SR | Error | % SR | Error |
| 1 | 69 | 3 | 51 | 3 |
| 2 | 80 | 3 | 57 | 6 |
| 3 | 86 | 1 | 89 | 1 |
| 4 | 81 | 4 | 67 | 8 |
| 5 | 91 | 1 | 91 | 1 |

(Standard error was calculated as SE = SD/√n where SD = standard deviation and n = number of external replicates)

The data clearly show that the addition of a rinse additive or a soil release polymer i.e. pre-condition compositions 2 and 4 respectively, significantly improves percentage soil removal. Further to this, with the addition of a lipid esterase in combination with either the rinse additive or soil release polymer i.e. pre-condition compositions 3 and 5 respectively result in higher percentage soil removal still. Thus, the inclusion of a lipid esterase in combination with either a rinse additive or soil release polymer during the rinse step of the pre-condition according to the present invention showed a surprising improvement in percentage soil reduction as compared to fabrics pre-conditioned without a lipid esterase.

### Examples 3-8

The following examples are of rinse additive compositions suitable for use in step (i);

| | 3 | 4 | 5 | 6 | 7 | 8 |
|---|---|---|---|---|---|---|
| | (wt %) | (wt %) | (wt %) | (wt %) | (wt %) | (wt %) |
| Silicone HC306 | 6.0 | 6.0 | 3.0 | 4.0 | 8.0 | 12.0 |
| PAMMAPTAC | 0.1 | 0.1 | 0.1 | 0.4 | 0.4 | 0.2 |
| Butyl carbitol | 3.0 | 3.0 | 4.0 | 0.0 | 0.0 | 0.0 |
| TAE₈₀ | 0.1 | 0.05 | 0.05 | 0.1 | 0.1 | 0.1 |
| Proxel ™ GXL | 0.02 | 0.02 | 0.02 | 0.02 | 0.02 | 0.02 |
| Perfume Best B | 0.5 | 0.5 | 0.5 | 0.4 | 0.4 | 0.4 |
| Water | 90.28 | 90.33 | 92.33 | 95.08 | 93.08 | 87.28 |

### Examples 9-27

The following examples are of laundry detergent compositions suitable for use in step (i) and (iii);

### Examples 9-14

Granular laundry detergent compositions designed for hand washing or top-loading washing machines may be added to sufficient water to form a paste for direct contact with the surface to be treated, forming a concentrated cleaning composition.

| | 9 | 10 | 11 | 12 | 13 | 14 |
|---|---|---|---|---|---|---|
| | (wt %) | (wt %) | (wt %) | (wt %) | (wt %) | (wt %) |
| Linear alkylbenzenesulfonate | 20 | 22 | 20 | 15 | 20 | 20 |
| C₁₂₋₁₄ Dimethylhydroxyethyl | 0.7 | 0.2 | 1 | 0.6 | 0.0 | 0 |
| ammonium chloride | | | | | | |
| AE3S | 0.9 | 1 | 0.9 | 0.0 | 0.5 | 0.9 |
| AE7 | 0.0 | 0.0 | 0.0 | 1 | 0.0 | 3 |
| Sodium tripolyphosphate | 5 | 0.0 | 4 | 9 | 2 | 0.0 |
| Zeolite A | 0.0 | 1 | 0.0 | 1 | 4 | 1 |
| 1.6 R Silicate (SiO₂:Na₂O at | 7 | 5 | 2 | 3 | 3 | 5 |
| ratio 1.6:1) | | | | | | |
| Sodium carbonate | 25 | 20 | 25 | 17 | 18 | 19 |
| Polyacrylate MW 4500 | 1 | 0.6 | 1 | 1 | 1.5 | 1 |
| Random graft copolymer¹ | 0.1 | 0.2 | 0.0 | 0.0 | 0.0 | 0.0 |
| Carboxymethyl cellulose | 1 | 0.3 | 1 | 1 | 1 | 1 |
| Stainzyme ® (20 mg active/g) | 0.1 | 0.2 | 0.1 | 0.2 | 0.1 | 0.1 |
| Bacterial protease (Savinase ®, | | | | | | |
| 32.89 mg active/g) | 0.1 | 0.1 | 0.1 | 0.1 | 0.1 | |
| Natalase ® (8.65 mg active/g) | 0.1 | 0.0 | 0.1 | 0.0 | 0.1 | 0.1 |
| Lipex ® (18 mg active/g) | 0.03 | 0.07 | 0.3 | 0.1 | 0.07 | 0.4 |
| Biotouch ® ROC (20 mg | 0.1 | 0.2 | 0.2 | 0.2 | 0.1 | 0.4 |
| active/g) | | | | | | |
| Fluorescent Brightener 1 | 0.06 | 0.0 | 0.06 | 0.18 | 0.06 | 0.06 |
| Fluorescent Brightener 2 | 0.1 | 0.06 | 0.1 | 0.0 | 0.1 | 0.1 |
| DTPA | 0.6 | 0.8 | 0.6 | 0.25 | 0.6 | 0.6 |
| MgSO₄ | 1 | 1 | 1 | 0.5 | 1 | 1 |
| Sodium Percarbonate | 0.0 | 5.2 | 0.1 | 0.0 | 0.0 | 0.0 |
| Sodium Perborate | 4.4 | 0.0 | 3.85 | 2.09 | 0.78 | 3.63 |
| Monohydrate | | | | | | |
| NOBS | 1.9 | 0.0 | 1.66 | 0.0 | 0.33 | 0.75 |
| TAED | 0.58 | 1.2 | 0.51 | 0.0 | 0.015 | 0.28 |
| Sulphonated zinc | 0.0030 | 0.0 | 0.0012 | 0.0030 | 0.0021 | 0.0 |
| phthalocyanine | | | | | | |
| S-ACMC | 0.1 | 0.0 | 0.0 | 0.0 | 0.06 | 0.0 |
| Direct Violet 9 | 0.0 | 0.0 | 0.0003 | 0.0005 | 0.0003 | 0.0 |
| Acid Blue 29 | 0.0 | 0.0 | 0.0 | 0.0 | 0.0 | 0.0003 |

| | |
|---|---|
| Sulfate/Moisture | Balance |

### Examples 15-20

Granular laundry detergent compositions designed for front-loading automatic washing machines may be added to sufficient water to form a paste for direct contact with the surface to be treated, forming a concentrated cleaning composition.

| | 15 | 16 | 17 | 18 | 19 | 20 |
|---|---|---|---|---|---|---|
| | (wt %) | (wt %) | (wt %) | (wt %) | (wt %) | (wt %) |
| Linear alkylbenzenesulfonate | 8 | 7.1 | 7 | 6.5 | 7.5 | 7.5 |
| AE3S | 0 | 4.8 | 0 | 5.2 | 4 | 4 |
| C12-14 Alkylsulfate | 1 | 0 | 1 | 0 | 0 | 0 |
| AE7 | 2.2 | 0 | 3.2 | 0 | 0 | 0 |
| C₁₀₋₁₂ Dimethyl | 0.75 | 0.94 | 0.98 | 0.98 | 0 | 0 |
| hydroxyethylammonium chloride | | | | | | |
| Crystalline layered silicate (δ- | 4.1 | 0 | 4.8 | 0 | 0 | 0 |
| Na₂Si₂O₅) | | | | | | |
| Zeolite A | 5 | 0 | 5 | 0 | 2 | 2 |
| Citric Acid | 3 | 5 | 3 | 4 | 2.5 | 3 |
| Sodium Carbonate | 15 | 20 | 14 | 20 | 23 | 23 |
| Silicate 2R (SiO₂:Na₂O at ratio | 0.08 | 0 | 0.11 | 0 | 0 | 0 |
| 2:1) | | | | | | |
| Soil release agent | 0.75 | 0.72 | 0.71 | 0.72 | 0 | 0 |
| Acrylic Acid/Maleic Acid | 1.1 | 3.7 | 1.0 | 3.7 | 2.6 | 3.8 |
| Copolymer | | | | | | |
| Carboxymethylcellulose | 0.15 | 1.4 | 0.2 | 1.4 | 1 | 0.5 |
| Bacterial protease (84 mg | | | | | | |
| active/g) | 0.2 | 0.2 | 0.3 | 0.15 | 0.12 | 0.13 |
| Stainzyme ® (20 mg active/g) | 0.2 | 0.15 | 0.2 | 0.3 | 0.15 | 0.15 |
| Lipex ® (18.00 mg active/g) | 0.05 | 0.15 | 0.1 | 0 | 0 | 0 |
| Natalase ® (8.65 mg active/g) | 0.1 | 0.2 | 0 | 0 | 0.15 | 0.15 |
| Celluclean ™ (15.6 mg active/g) | 0 | 0 | 0 | 0 | 0.1 | 0.1 |
| Biotouch ® ROC (20 mg active/g) | 0.2 | 0.1 | 0.2 | 0.2 | 0.2 | 0.2 |
| TAED | 3.6 | 4.0 | 3.6 | 4.0 | 2.2 | 1.4 |
| Percarbonate | 13 | 13.2 | 13 | 13.2 | 16 | 14 |
| Na salt of Ethylenediamine-N,N′- | 0.2 | 0.2 | 0.2 | 0.2 | 0.2 | 0.2 |
| disuccinic acid, (S,S) isomer | | | | | | |
| (EDDS) | | | | | | |
| Hydroxyethane di phosphonate | 0.2 | 0.2 | 0.2 | 0.2 | 0.2 | 0.2 |
| (HEDP) | | | | | | |
| MgSO₄ | 0.42 | 0.42 | 0.42 | 0.42 | 0.4 | 0.4 |
| Perfume | 0.5 | 0.6 | 0.5 | 0.6 | 0.6 | 0.6 |
| Suds suppressor agglomerate | 0.05 | 0.1 | 0.05 | 0.1 | 0.06 | 0.05 |
| Soap | 0.45 | 0.45 | 0.45 | 0.45 | 0 | 0 |
| Sulphonated zinc phthalocyanine | 0.0007 | 0.0012 | 0.0007 | 0 | 0 | 0 |
| (active) | | | | | | |
| S-ACMC | 0.01 | 0.01 | 0 | 0.01 | 0 | 0 |
| Direct Violet 9 (active) | 0 | 0 | 0.0001 | 0.0001 | 0 | 0 |

| | |
|---|---|
| Sulfate/Water & Miscellaneous | Balance |

Any of the above compositions is used to launder fabrics in the second step at a concentration of 7000 to 10000 ppm in water, 20-90° C., and a 5:1 water:cloth ratio. The typical pH is about 10. The fabrics are then dried. In one aspect, the fabrics are actively dried using a dryer. In one aspect, the fabrics are actively dried using an iron. In another aspect, the fabrics are merely allowed to dry on a line wherein they are exposed to air and optionally sunlight.

### Examples 21-26

### Heavy Duty Liquid Laundry Detergent Compositions

| | 21 | 22 | 23 | 24 | 25 | 26 |
|---|---|---|---|---|---|---|
| | (wt %) | (wt %) | (wt %) | (wt %) | (wt %) | (wt %) |
| AES C₁₂₋₁₅ alkyl | 11 | 10 | 4 | 6.32 | 0 | 0 |
| ethoxy (1.8) | | | | | | |
| sulfate | | | | | | |
| AE3S | 0 | 0 | 0 | 0 | 2.4 | 0 |
| Linear alkyl | 1.4 | 4 | 8 | 3.3 | 5 | 8 |
| benzene | | | | | | |
| sulfonate | | | | | | |
| HSAS | 3 | 5.1 | 3 | 0 | 0 | 0 |
| Sodium formate | 1.6 | 0.09 | 1.2 | 0.04 | 1.6 | 1.2 |
| Sodium | 2.3 | 3.8 | 1.7 | 1.9 | 1.7 | 2.5 |
| hydroxide | | | | | | |
| Monoethanol- | 1.4 | 1.49 | 1.0 | 0.7 | 0 | 0 |
| amine | | | | | | |
| Diethylene | 5.5 | 0.0 | 4.1 | 0.0 | 0 | 0 |
| glycol | | | | | | |
| AE9 | 0.4 | 0.6 | 0.3 | 0.3 | 0 | 0 |
| AE7 | 0 | 0 | 0 | 0 | 2.4 | 6 |
| Chelant | 0.15 | 0.15 | 0.11 | 0.07 | 0.5 | 0.11 |
| Citric Acid | 2.5 | 3.96 | 1.88 | 1.98 | 0.9 | 2.5 |
| C₁₂₋₁₄ dimethyl | 0.3 | 0.73 | 0.23 | 0.37 | 0 | 0 |
| Amine Oxide | | | | | | |
| C₁₂₋₁₈ Fatty Acid | 0.8 | 1.9 | 0.6 | 0.99 | 1.2 | 0 |
| 4-formyl- | 0 | 0 | 0 | 0 | 0.05 | 0.02 |
| phenylboronic | | | | | | |
| acid | | | | | | |
| Borax | 1.43 | 1.5 | 1.1 | 0.75 | 0 | 1.07 |
| Ethanol | 1.54 | 1.77 | 1.15 | 0.89 | 0 | 3 |
| Ethoxylated | 0.3 | 0.33 | 0.23 | 0.17 | 0.0 | 0.0 |
| (EO₁₅) | | | | | | |
| tetraethylene | | | | | | |
| pentamine | | | | | | |
| Ethoxylated | 0.8 | 0.81 | 0.6 | 0.4 | 1 | 1 |
| hexamethylene | | | | | | |
| diamine | | | | | | |
| 1,2-Propanediol | 0.0 | 6.6 | 0.0 | 3.3 | 0.5 | 2 |
| Bacterial protease | 0.8 | 0.6 | 0.7 | 0.9 | 0.7 | 0.6 |
| (40.6 mg active/g) | | | | | | |
| Mannaway ® | 0.07 | 0.05 | 0.045 | 0.06 | 0.04 | 0.045 |
| (25 mg active/g) | | | | | | |
| Stainzyme ® | 0.3 | 0.2 | 0.3 | 0.1 | 0.2 | 0.4 |
| (15 mg active/g) | | | | | | |
| Natalase ® | 0 | 0.2 | 0.1 | 0.15 | 0.07 | 0 |
| (29 mg active/g) | | | | | | |
| Lipex ® (18 mg | 0.4 | 0.2 | 0.3 | 0.1 | 0.2 | 0 |
| active/g) | | | | | | |
| Biotouch ® ROC | 0.2 | 0.1 | 0.2 | 0.2 | 0.1 | 0.1 |
| (20 mg active/g) | | | | | | |
| Liquitint ® | 0.006 | 0.002 | 0 | 0 | 0 | 0.002 |
| Violet CT (active) | | | | | | |
| S-ACMC | — | — | 0.01 | 0.05 | 0.01 | 0.02 |
| Water, perfume, | Balance | | | | | |
| dyes & other | | | | | | |
| components | | | | | | |

### Example 27

This composition may be enclosed in a polyvinyl alcohol pouch.

| | 27 |
|---|---|
| | (wt %) |
| Alkylbenzene sulfonic acid | 21.0 |
| C₁₄₋₁₅ alkyl 8-ethoxylate | 18.0 |
| C₁₂₋₁₈ Fatty acid | 15.0 |
| Bacterial protease (40.6 mg active/g) | 1.5 |
| Natalase ® (29 mg active/g) | 0.2 |
| Mannanase (Mannaway ®, 11 mg active/g) | 0.1 |
| Xyloglucanase (Whitezyme ®, 20 mg active/g) | 0.2 |
| Biotouch ® ROC (20mg active/g) | 0.2 |
| A compound having the following general | 2.0 |
| structure: bis((C₂H₅O)(C₂H₄O)n)(CH₃)—N⁺— | |
| CₓH₂ₓ—N⁺—(CH₃)-bis((C₂H₅O)(C₂H₄O)n), | |
| wherein n = from 20 to 30, and x = from 3 to | |
| 8, or sulphated or sulphonated variants thereof | |
| Ethoxylated Polyethylenimine ² | 0.8 |
| Hydroxyethane diphosphonate (HEDP) | 0.8 |
| Fluorescent Brightener 1 | 0.2 |
| Solvents (1,2 propanediol, ethanol), stabilizers | 15.0 |
| Hydrogenated castor oil derivative structurant | 0.1 |
| Perfume | 1.6 |
| Core Shell Melamine-formaldehyde | 0.10 |
| encapsulate of perfume | |
| Ethoxylated thiophene Hueing Dye | 0.004 |
| Buffers (sodium hydroxide, | To pH 8.2 |
| Monoethanolamine) | |
| Water* and minors (antifoam, aesthetics) | To 100% |

*Based on total cleaning and/or treatment composition weight, a total of no more than 7% water
1 Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.
² Polyethyleneimine (MW = 600) with 20 ethoxylate groups per —NH.
*Remark: all enzyme levels expressed as % enzyme raw material

### Raw Materials and Notes for Composition Examples 3-8

Silicone HC306 supplied by Wacker Chemical Corporation, Adrian, Mich., USA
Poly(acrylamide-methacrylamidopropyltrimethyl ammonium chloride (PAMMAPTAC) supplied by
Butyl carbitol, supplied by
Tallow alkyl ethoxylated alcohol with an average degree of ethoxylation of 80 supplied by
Proxel™ GXL supplied by
Perfume Best B supplied by

### Raw Materials and Notes for Composition Examples 9-27

Linear alkylbenzenesulfonate having an average aliphatic carbon chain length C₁₁-C₁₂ supplied by Stepan, Northfield, Ill., USA
C₁₂₋₁₄ Dimethylhydroxyethyl ammonium chloride, supplied by Clariant GmbH, Sulzbach, Germany
AE3S is C₁₂₋₁₅ alkyl ethoxy (3) sulfate supplied by Stepan, Northfield, Ill., USA
AE7 is C₁₂₋₁₅ alcohol ethoxylate, with an average degree of ethoxylation of 7, supplied by Huntsman, Salt Lake City, Utah, USA
AE9 is C₁₂₋₁₃ alcohol ethoxylate, with an average degree of ethoxylation of 9, supplied by Huntsman, Salt Lake City, Utah, USA
HSAS is a mid-branched primary alkyl sulfate with carbon chain length of about 16-17
Sodium tripolyphosphate is supplied by Rhodia, Paris, France
Zeolite A is supplied by Industrial Zeolite (UK) Ltd, Grays, Essex, UK
1.6R Silicate is supplied by Koma, Nestemica, Czech Republic
Sodium Carbonate is supplied by Solvay, Houston, Tex., USA
Polyacrylate MW 4500 is supplied by BASF, Ludwigshafen, Germany
Carboxymethyl cellulose is Finnfix® V supplied by CP Kelco, Arnhem, Netherlands
Suitable chelants are, for example, diethylenetetraamine pentaacetic acid (DTPA) supplied by Dow Chemical, Midland, Mich., USA or Hydroxyethane di phosphonate (HEDP) supplied by Solutia, St Louis, Mo., USA Bagsvaerd, Denmark
Savinase®, Natalase®, Stainzyme®, Lipex®, Celluclean™, Mannaway® and Whitezyme® are all products of Novozymes, Bagsvaerd, Denmark.
Biotouch® ROC is a product of AB Enzymes, Darmstadt, Germany.
Bacterial protease (examples 8-13) described in U.S. Pat. No. 6,312,936 B1 supplied by Genencor International, Palo Alto, Calif., USA
Bacterial protease (examples 14-20) described in U.S. Pat. No. 4,760,025 is supplied by Genencor International, Palo Alto, Calif., USA
Fluorescent Brightener 1 is Tinopal® AMS, Fluorescent Brightener 2 is Tinopal® CBS-X, Sulphonated zinc phthalocyanine and Direct Violet 9 is Pergasol® Violet BN-Z all supplied by Ciba Specialty Chemicals, Basel, Switzerland
Sodium percarbonate supplied by Solvay, Houston, Tex., USA
Sodium perborate is supplied by Degussa, Hanau, Germany
NOBS is sodium nonanoyloxybenzenesulfonate, supplied by Future Fuels, Batesville, Ark., USA
TAED is tetraacetylethylenediamine, supplied under the Peractive® brand name by Clariant GmbH, Sulzbach, Germany
S-ACMC is carboxymethylcellulose conjugated with C.I. Reactive Blue 19, sold by Megazyme, Wicklow, Ireland under the product name AZO-CM-CELLULOSE, product code S-ACMC.
Soil release agent is Repel-o-tex® PF, supplied by Rhodia, Paris, France
Acrylic Acid/Maleic Acid Copolymer is molecular weight 70,000 and acrylate:maleate ratio 70:30, supplied by BASF, Ludwigshafen, Germany
Na salt of Ethylenediamine-N,N′-disuccinic acid, (S,S) isomer (EDDS) is supplied by Octel, Ellesmere Port, UK
Hydroxyethane di phosphonate (HEDP) is supplied by Dow Chemical, Midland, Mich., USA
Suds suppressor agglomerate is supplied by Dow Corning, Midland, Mich., USA
HSAS is mid-branched alkyl sulfate as disclosed in U.S. Pat. No. 6,020,303 and U.S. Pat. No. 6,060,443
C₁₂₋₁₄ dimethyl Amine Oxide is supplied by Procter & Gamble Chemicals, Cincinnati, Ohio, USA
Liquitint® Violet CT is supplied by Milliken, Spartanburg, S.C., USA.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of laundering a fabric, comprising the steps of;
i) contacting a fabric with an aqueous wash liquor comprising a detergent composition, the detergent composition comprising a surfactant and wherein the wash liquor comprises from about 0.05 to about 4 g/l of a surfactant; and
ii) in a first rinse step, contacting the fabric from step (i) with an aqueous rinse liquor;
iii) in a second rinse step, contacting the fabric from step (ii) with a second aqueous rinse liquor comprising a lipid esterase;
iv) drying the fabric;
v) contacting the fabric from step (iv) with a soil;
vi) contacting the fabric from step (v) with an aqueous wash liquor comprising a detergent composition wherein the detergent composition comprises a surfactant, the wash liquor comprising from about 0.05 to about 4 g/l of a surfactant.

2. A method according to claim 1, wherein the lipid esterase comprises at least a first and a second lipid esterase.

3. A method according to claim 1 wherein the fabric in step (i) and/or step (vi) is contacted with a laundry detergent composition in a wash cycle of an automatic washing machine and wherein the length of the wash cycle is at least about 30 seconds, or even at least about 3 mins, or even at least about 6 mins, but no more than about 30 mins, or even no more than about 45 mins, or even no more than about 1 hour.

4. A method according to claim 1, wherein the fabric in step (i) and/or step (vi) is contacted with a laundry detergent composition in a wash cycle of an automatic washing machine and wherein the wash cycle is run at a temperature of between about 5° C. and about 50° C.

5. A method according to claim 4, wherein the wash cycle is run at a temperature of between about 10° C. and about 30° C.

6. A method according to claim 1, wherein the lipid esterase is selected from classes E.C. class 3.1.1.1, E.C. class 3.1.1.3, E.C. class 3.1.1.74 and a combination thereof.

7. A method according to claim 6, wherein the lipid esterase is selected from classes E.C. 3.1.1.3, E.C. 3.1.1.1 and a combination thereof, preferably E.C.3.1.1.3.

8. A method according to any claim 1 wherein the lipid esterase comprises a variant (i) having at least 90% sequence identity to wild-type lipase from Thermomyces lanuginosus and having sequence substitutions T231R and N233R, or (ii) a variant cutinase having a) an amino acid sequence with above 80% homology with SEQ ID NO: 1, b) compared to SEQ ID NO: 1 comprises substitution of amino acid residue A4, A88, N91, A130, Q139, T166, L167, 1168, 1169 or R189, or an amino acid substitution Q1C/L, L2K/Q/V, G8D, S11T, N15D, A16T, T29M/I/C, V38H, N44D, S48E/K, H49Y, L66I, S116K, S119P, G120D, T164S, L174F or I178V and c) is more thermostable than the cutinase having the amino acid sequence SEQ ID NO: 1, and d) comprises the following amino acid substitutions compared to SEQ ID NO: 1: G8D+N15D+S48E+A88H+N91H+A130V+R189V, or a combination thereof, wherein SEQ ID NO: 1 is the following amino acid sequence: Gln Leu Gly Ala Ile Glu Asn Gly Leu Glu Ser Gly Ser Ala Asn Ala Cys Pro Asp Ala Ile Leu Ile Phe Ala Arg Gly Ser Thr Glu Pro Gly Asn Met Gly Ile Thr Val Gly Pro Ala Leu Ala Asn Gly Leu Glu Ser His Ile Arg Asn Ile Trp Ile Gln Gly Val Gly Gly Pro Tyr Asp Ala Ala Leu Ala Thr Asn Phe Leu Pro Arg Gly Thr Ser Gln Ala Asn Ile Asp Glu Gly Lys Arg Leu Phe Ala Leu Ala Asn Gln Lys Cys Pro Asn Thr Pro Val Val Ala Gly Gly Tyr Ser Gln Gly Ala Ala Leu Ile Ala Ala Ala Val Ser Glu Leu Ser Gly Ala Val Lys Glu Gln Val Lys Gly Val Ala Leu Phe Gly Tyr Thr Gln Asn Leu Gln Asn Arg Gly Gly Ile Pro Asn Tyr Pro Arg Glu Arg Thr Lys Val Phe Cys Asn Val Gly Asp Ala Val Cys Thr Gly Thr Leu Ile Ile Thr Pro Ala His Leu Ser Tyr Thr Ile Glu Ala Arg Gly Glu Ala Ala Arg Phe Leu Arg Asp Arg Ile Arg Ala.

9. A method according to claim 1 wherein in step (iii) the fabric is contacted with a lipid esterase the lipid esterase being present in the aqueous rinse liquor at a concentration of between about 30 and about 55,000 ng enzyme/g fabric.

10. A method according to claim 1 wherein the ratio of surfactant to fabric on a weight to weight basis in step (i) and/or step (vi) is of from about 1:150 to about 1:500.

11. A method according to claim 1 wherein the aqueous rinse solution in the second aqueous rinse step (iii) additionally comprises a soil release polymer.

12. A method according to claim 11 wherein the soil release polymer comprises a terephthalate soil release polymer.

13. A method according to claim 1, wherein the second rinse step is the final rinse step before the fabric is dried in step (iv).

14. A method according to claim 1, wherein the detersive surfactant comprises an anionic surfactant, selected from linear alkyl benzene sulfonate, alkoxylated anionic surfactant, and a combination thereof.

15. A method according to claim 1, wherein the laundry detergent composition of step (i) and/or step (vi) comprises a hueing agent, a polymer or a combination thereof.

16. A method according to claim 1, wherein the laundry detergent composition of step (i) and/or step (vi) comprises from about 0 wt % to about 10 wt % zeolite builder on an anhydrous basis, from about 0 wt % to about 10 wt % phosphate builder, or a combination thereof.


SEQUENCE LISTING (1 sequence)

SEQ ID NO: 1 - protein, 194 aa, Humicola insolens
QLGAIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA